(19) United States
(12) Patent Application Publication
(10) Pub. No.: US 2011/0152099 A1
(43) Pub. Date: Jun. 23, 2011

(54) BIOACTIVE COMPOUNDS OF ASCOPHYLLUM NODOSUM AND THEIR USE FOR ALLEVIATING SALT-INDUCED STRESS IN PLANTS

(76) Inventors: Balakrishnan Prithiviraj, Truro (CA); Pragya Kant, Truro (CA); Jithesh M. Narayanan, Truro (CA); Wajahatullah Khan, Truro (CA); Simon Hankins, Clayton (GB); William Neily, Paradise (CA); Alan T. Critchley, Bedford (CA); James S. Craigie, Halifax (CA)

(21) Appl. No.: 12/936,074

(22) PCT Filed: Apr. 1, 2009
(86) PCT No.: PCT/CA2009/000419

§ 371 Date: Jan. 21, 2011

Related U.S. Application Data
(60) Provisional application No. 61/041,369, filed on Apr. 1, 2008.

(51) Int. Cl. A01N 65/03; C07J 9/00; C07C 53/126; A01N 37/00; A01N 31/06; A01N 27/00; A01P 21/00; C07C 11/00

(52) U.S. Cl. 504/142; 504/118; 552/544; 554/1; 585/16

(57) ABSTRACT

A process for extraction of bioactive organic compounds from alkali extracts of Ascophyllum nodosum. Organic solvent extracts of A. nodosum namely; methanol, chloroform and ethylacetate, were found to alleviate salt induced stress in plants. This effect was due to alteration in the expression of a specific subset of genes induced by compounds present in A. nodosum extracts.

### FIELD OF THE INVENTION

The invention relates to compounds and methods for alleviating salt-induced stress in plants. More specifically, the invention relates to compounds and extracts derived from Ascophyllum nodosum, methods of their production, and their use for the alleviating salt-induced stress in plants.

### BACKGROUND OF THE INVENTION

Plant growth and productivity is severely affected by various abiotic stresses, like salinity, temperature extremes and drought. Of these, soil salinization is a major constraint in food production on otherwise potentially arable lands, and is thus of particular concern.

Efforts have been made to overcome the problems associated with high soil salinity through the use of plants with a higher level of tolerance to salt stress, by modifying genes encoding different proteins developed through biotechnological approaches. This approach has not yet translated into marketable crop varieties despite several years of research. Further, there has been heightened consumer sensitivity to genetically modified (GM) foods. Accordingly, there continues to be the need for a way to address the problems associated with high soil salinity.

Ascophyllum nodosum (rockweed), a brown algae that grows along the Canadian Atlantic coast, has acquired special mechanisms of salt tolerance, possibly by synthesis of bioactive compounds. Accordingly, the present inventors have sought to develop an alternative approach for alleviating negative effects of salt stress on salt sensitive plants through the isolation of such bioactive compounds.

### SUMMARY OF THE INVENTION

Accordingly, an object of the present invention is to provide a means for alleviating salt-induced stress in plants.

According to an aspect of the present invention, there is provided a process for preparing an organic extract useful as a treatment for inducing salinity tolerance in plants, said process comprising the steps of: (a) suspending dried A. nodosum in methanol, (b) mixing the suspension, and (c) separating any remaining solid material from the resulting methanol extract, wherein the methanol extract is useful as a treatment for inducing salinity tolerance in plants.

In an embodiment, the process may further comprise the steps of: (d) removing the solvent from said methanol extract to form a dried residual, (e) resuspending the dried residual from said methanol extract in water, (f) adding chloroform to the resuspended residual from the methanol extract, (g) mixing and allowing the phases to separate into water and chloroform extracts, (h) collecting the chloroform extract, and (i) removing the solvent from said chloroform extract to form a dried residual, wherein the dried residual of said chloroform extract is useful as a treatment for inducing salinity tolerance in plants. In a further embodiment, the process may additionally comprise the steps: (j) resuspending the dried residual from said chloroform extract in water, (k) adding ethyl acetate to the resuspended residual from the chloroform extract, (l) mixing and allowing the phases to separate into water and ethyl acetate extracts, (m) collecting the ethyl acetate extract, and (n) removing the solvent from said ethyl acetate extract to form a dried residual, wherein the dried residual of said ethyl acetate extract is useful as a treatment for inducing salinity tolerance in plants.

In further embodiments, the A. nodosum is suspended in the methanol in a ratio of A. nodosum to methanol from about 1:1 to about 1:50 volume/volume. In a preferred embodiment, the A. nodosum is suspended in the methanol in a ratio of A. nodosum to methanol of 1:3 volume/volume.

In yet further embodiments, it may be preferred that steps (f)-(h) be repeated up to 3 times. Similarly, it may be preferred that steps (k)-(m) be repeated up to 3 times.

As a further aspect of the invention, there is provided a method of inducing salinity tolerance in plants, comprising obtaining an organic extract as defined in the above process, and administering the organic extract to a plant under salt stress in an amount effective to ameliorate the salt stress in said plant.

Also provided, as an aspect, is the use of an organic extract as defined in the above process for inducing salinity tolerance in plants.

As another aspect of the invention, there is provided a composition for inducing salinity tolerance in plants, comprising as active agent at least one phytosterol, fungal sterol, terpenoid or fatty acid, or combinations thereof, derived from Ascophyllum nodosum.

In the above composition, the fungal sterol may be derived from Mycosphaerella ascophylli.

In an embodiment of the above composition, the phytosterol is fucosterol.

As a further aspect, there is provided a method of inducing salinity tolerance in plants, comprising administering a composition as defined above to a plant under salt stress in an amount effective to ameliorate said salt stress in said plant.

Also provided is the use of a composition as defined above for inducing salinity tolerance in plants.

As an additional aspect, there is provided a method of inducing salinity tolerance in plants, comprising extracting at least one phytosterol, fungal sterol, terpenoid or fatty acid, or combinations thereof from Ascophyllum nodosum, and administering said organic extract to a plant under salt stress in an amount effective to ameliorate said salt stress in said plant.

In a further aspect of the invention, there is provided a composition of matter comprising at least one phytosterol, fungal sterol, terpenoid, fatty acid, or combinations thereof, from Ascophyllum nodosum, for alleviating salinity stress in plants. In an embodiment of the composition of matter, the phytosterol, fungal sterol, terpenoid, fatty acid, or combinations thereof elicit coordinated expression of multiple genes in the plant to induce salinity tolerance.

The invention also provides a composition useful as a treatment for inducing salinity tolerance in plants, obtained according to a process including (a) suspending dried A. nodosum or extracts of A. nodosum in methanol, (b) mixing the suspension, and (c) separating any remaining solid material from the resulting methanol extract, wherein the methanol extract is provided as a composition useful as a treatment for inducing salinity tolerance in plants.

In an embodiment, the methanol extracts described above may have the solvent at least partially removed such that the extracted organic material, e.g. including one or more of phytosterols, fungal sterols, terpenoids, fatty acids, and combinations thereof, can be used in a plant or seed treatment.

In other embodiments, the methanol extract may be further processed by (d) removing the solvent from the methanol extract to form a dried residual, (e) resuspending the dried residual from the methanol extract in water, (f) adding chloroform to the resuspended residual from the methanol extract, (g) mixing and allowing the phases to separate into water and chloroform extracts, (h) collecting the chloroform extract, and (i) removing the solvent from said chloroform extract to form a dried residual, wherein the dried residual of the chloroform extract is provided as a composition useful for inducing salinity tolerance in plants. Further processing of the dried residual of the chloroform extract can also be undertaken by (j) resuspending the dried residual from the chloroform extract in water, (k) adding ethyl acetate to the resuspended residual from the chloroform extract, (l) mixing and allowing the phases to separate into water and ethyl acetate extracts, (m) collecting the ethyl acetate extract, and (n) removing the solvent from the ethyl acetate extract to form a dried residual, wherein the dried residual of the ethyl acetate extract is provided as a composition useful for inducing salinity tolerance in plants.

Compositions as described herein can be formulated for use, for instance, as a liquid for spray or root irrigation, or as a solid for seed treatment. Solid and liquid carriers useful in preparing such formulations will be known to those skilled in the art, and can accordingly be used in formulations of the present invention.

### BRIEF DESCRIPTION OF THE DRAWINGS

These and other features, aspects and advantages of the present invention will become better understood with regard to the following description and accompanying drawings wherein:

FIG. 1 is a schematic representation of the extraction of organic compounds of A. nodosum;

FIG. 2 shows the results of NMR analysis of the organic extracts of A. nodosum;

FIG. 3 shows a graphical representation of the results of testing organic extracts of A. nodosum. As shown, organic extracts of A. nodosum alleviate the negative effect of salt on fresh weight in A. thaliana;

FIG. 4 shows a graphical representation of the results of testing organic extracts of A. nodosum. As shown, organic extracts of A. nodosum alleviate the negative effect of salt on number of leaves in A. thaliana;

FIG. 5 shows a graphical representation of the results of testing organic extracts of A. nodosum. As shown, organic extracts of A. nodosum alleviate the negative effect of salt on leaf area in A. thaliana;

FIG. 6 shows a graphical representation of the results of testing organic extracts of A. nodosum. As shown, organic extracts of A. nodosum alleviate the negative effect of salt on plant height in A. thaliana;

FIG. 7 shows pictorial results of testing organic extracts of A. nodosum. As shown in the photographs, organic extracts of A. nodosum alleviates salt-induced stress in A. thaliana;

FIG. 8 depicts the results of testing the effect of organic extracts of A. nodosum on the expression of stress induced genes in A. thaliana, up arrows indicate increased expression, down arrows indicate lowered expression, and horizontal arrows indicate little or no change in expression;

FIG. 9 shows the results of testing the effect of organic compounds of A. nodosum on the Na⁺ uptake in A. thaliana;

FIG. 10 shows volcano plots of gene expression on Day 1 and Day 5 with organic extracts of A. nodosum, showing that the organic extracts affect gene expression and make the plant resistant to salinity stress;

FIG. 11 depicts venn diagrams illustrating that organic compounds of A. nodosum elicit specific stress tolerance response by up or down regulating specific sets of genes;

FIG. 12 shows the results of analysing expression levels by RT-PCR upon treatment with ethyl acetate extract fractions on Day 1 and Day 5. As observed, organic compounds of A. nodosum elicit specific stress tolerance responses by up or down regulating specific sets of genes;

FIG. 13 shows the results of testing catalase activity in lettuce after 24 h under 100 mM NaCl and 150 mM NaCl conditions;

FIG. 14 shows the results of testing catalase activity in lettuce after 48 h under 100 mM NaCl and 150 mM NaCl conditions;

FIG. 15 shows the results of measuring percentage leaf area as affected by salt stress in Lettuce under 100 mM NaCl and 150 mM NaCl conditions;

FIG. 16 shows the results of measuring chlorophyll content in Lettuce after 48 h under 100 mM NaCl and 150 mM NaCl conditions;

FIG. 17 shows the results of measuring chlorophyll content in sugarbeet after 48 h under 100 mM NaCl and 150 mM NaCl conditions;

FIG. 18 shows the results of testing fucosterol and different organic extracts of A. nodosum on root length. The code numbers in the figure refers to different organic extracts. As observed, plants treated with fucosterol and extract RS5-45C have a longer root length than untreated plants under salt stress (compared to +Na); and

FIG. 19 shows the reduced root Na⁺ uptake under hydrophonics system (ion-exclusion).

### DETAILED DESCRIPTION

The present inventors have identified organic compounds and extracts that alleviate salt stress in plants. This finding has significance for a variety of economically important crops encompassing major plant groups, including cereals (including but not limited to barley, wheat, rice, corn, oats) legumes (including but not limited to pea, mungbean, soybean), brassicas (including but not limited to cauliflower, broccoli, canola, mustard, rapeseed), tubers (including but not limited to beets, potatoes, carrots), and vegetables (including but not limited to tomato, cucumber, lettuce, pepper).

The compounds and extracts are derived from the brown intertidal alga Ascophyllum nodosum. This alga is known to have a systemic association with at least one fungus (in particular Mycosphaerella ascophylli) which grows in and amongst the internal tissues of the seaweed and is therefore inseparable. Accordingly, compounds and extracts of A. nodosum as described herein may also include compounds of fungal origin by virtue of the natural fungal associations with A. nodosum.

The fraction of the extract which is beneficial in providing salinity tolerance in land plants has been found to contain predominantly seaweed phytosterols, terpenoids and fatty acids. The extracts may also comprise fungal sterols. In an embodiment the phytosterol is a fucosterol.

Fucosterol (24-ethylidene cholesterol), has the following chemical structure:

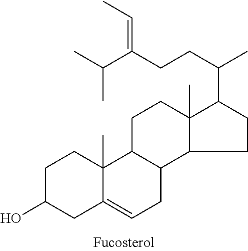

The present invention is particularly advantageous over the prior art since abiotic stress tolerance, including salinity stress tolerance, is imparted by multiple genes (oligogenic). Currently there are limitations to the number of genes that can be efficiently introgressed by genetic modification (transgenic approach) of plants and therefore inducing tolerance to abiotic stresses via the GM approach is limited. Further, chemicals in A. nodosum extracts also repress the expression of a number of genes that ultimately leads to salinity tolerance. Taken together, the organic compounds and extracts of A. nodosum disclosed herein can be used as an effective alternative to the GMO approach. It is also anticipated that this approach will have more consumer acceptance, and reduce possible ecological damage to the environment by GMOs.

In an embodiment of the invention, the extraction is carried out as follows: dry A. nodosum extract powder or dry A. nodosum powder is suspended in 100% methanol in a ratio of from about 1:1 to 1:50 volume/volume of powder to methanol, more preferably in a ratio of 1:3 powder to methanol, and mixed (e.g. by vortexing for 10 minutes at room temperature). The extract is then suspended in water (approximately 1:1 to 1:50 of the volume of the original A. nodosum powder, more preferably in a ratio of 1:3) and phase partitioned with chloroform (approximately 1:1 to 1:50 of the volume of the original A. nodosum powder, more preferably in a ratio of 1:3), preferably more than once and more preferably three times. The chloroform fractions are combined to provide the chloroform fraction (the analysis of which is discussed in further detail below). The remaining aqueous fraction is phase partitioned with ethyl acetate (approximately 1:1 to 1:50 of the volume of the original A. nodosum powder, more preferably in a ratio of 1:3), preferably more than once and more preferably three times. The ethyl acetate fractions are combined, the solvent evaporated and the residual solid formed the ethyl acetate fraction (the analysis of which is discussed in further detail below). Each of the fractions used in the experiments below was dissolved in methanol.

Experiments

Extraction of Bioactive Compounds from Alkali Extracts of A. nodosum

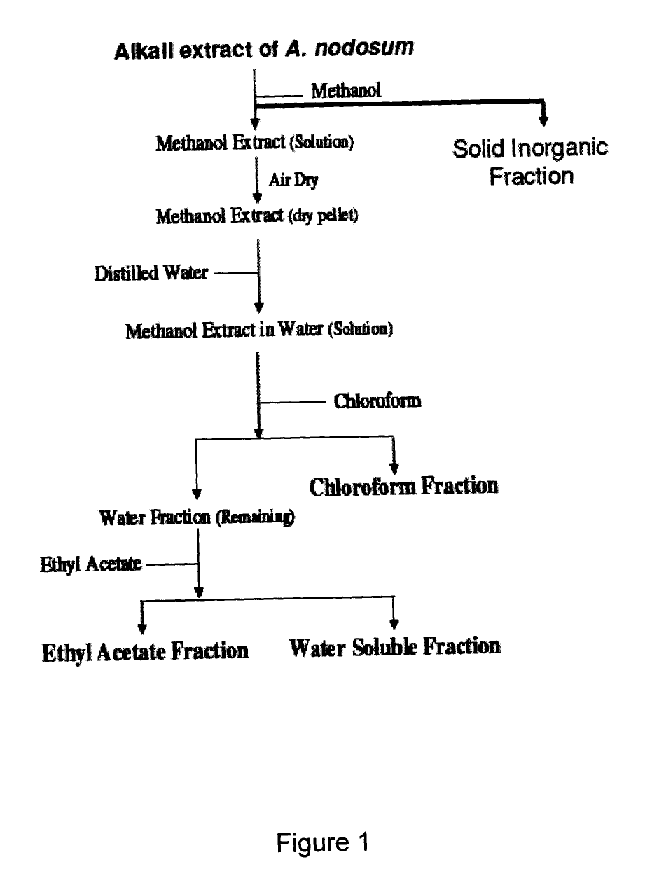

The schematic representation of the extraction protocol is presented as FIG. 1. Briefly, the alkali extract of A. nodosum was extracted in three volumes of HPLC grade methanol. The extract was centrifuged and the supernatant was dried to a pellet. The pellet was suspended in distilled water and sequentially fractionated with Chloroform and then Ethyl acetate. The resulting extracts were dried and suspended in pure methanol and used.

Nuclear Magnetic Resonance (NMR) Analysis of the Organic Extracts of A. nodosum

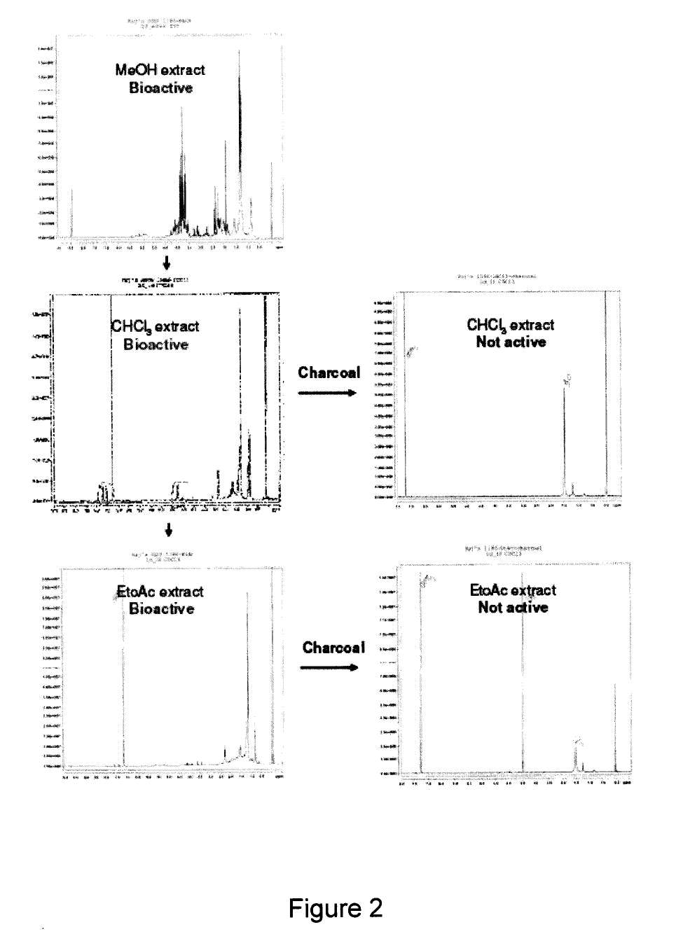

An NMR analysis revealed the presence of organic compounds, and predominantly fatty acids and sterols in the extracts (FIG. 2).

Organic Extracts of A. nodosum Alleviate Salt Induced Stress in Plants

Arabidopsis thaliana plants were grown in the green house at 22±2° C. under long day photo period (16 h light/8 h dark). Two-week-old plants were treated with 150 mM NaCl by flooding the roots (at the rate of 25 ml/plant). Twenty four hours after the salt treatment, plants were treated with organic extracts (methanol, chloroform and ethyl acetate) of A. nodosum at the rate of 25 ml/plant of a solution containing about 1 mg/liter of organic compounds. The extract treatment was repeated once after seven days. Observations on plant height, number of leaves, leaf area and fresh weight were recorded after one month of the salt treatment. The changes in the expression of stress inducible genes were studied at five days after treatment.

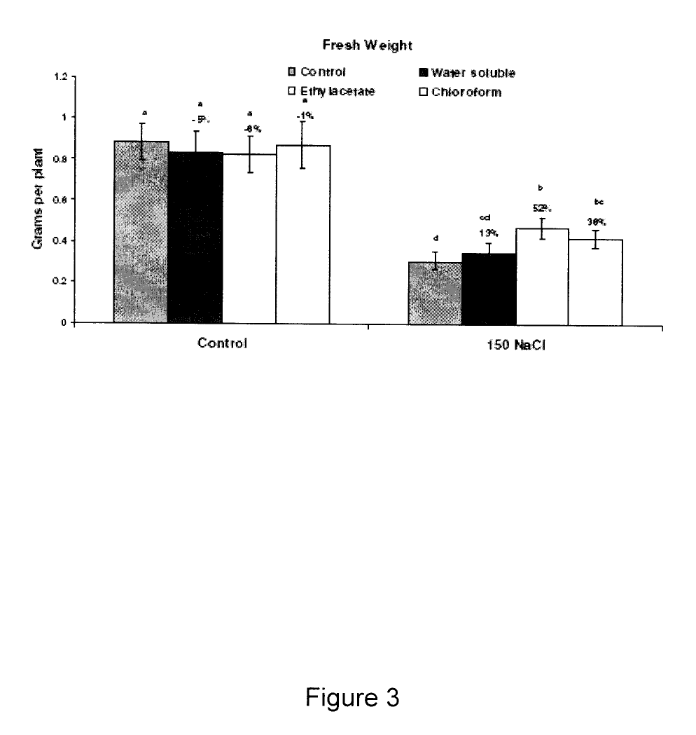

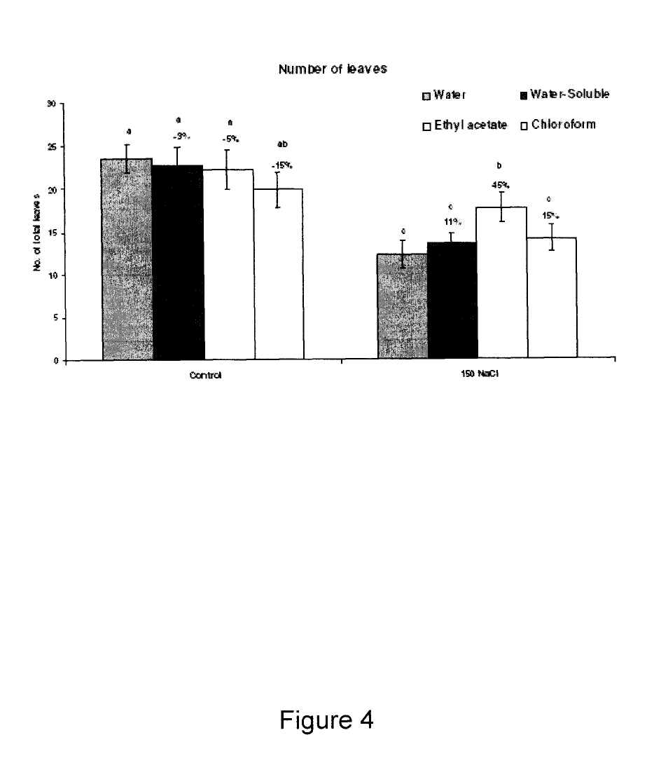

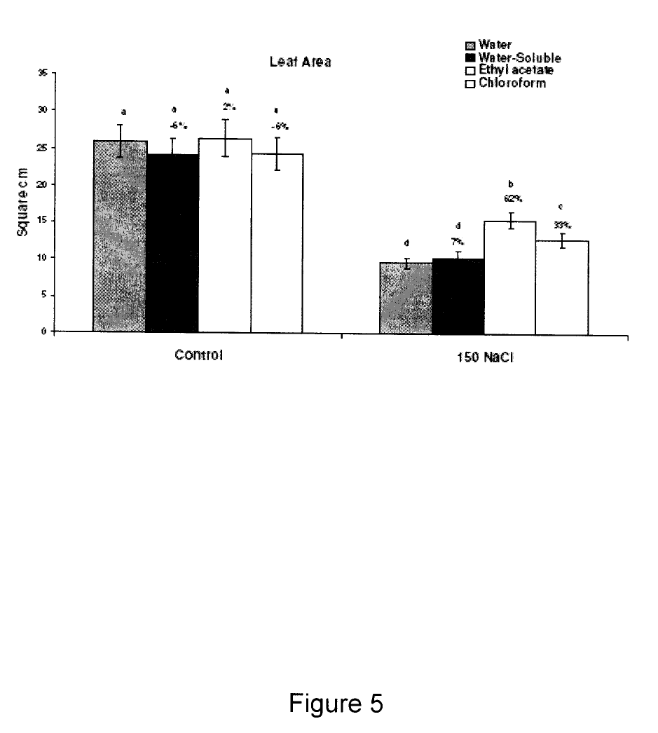

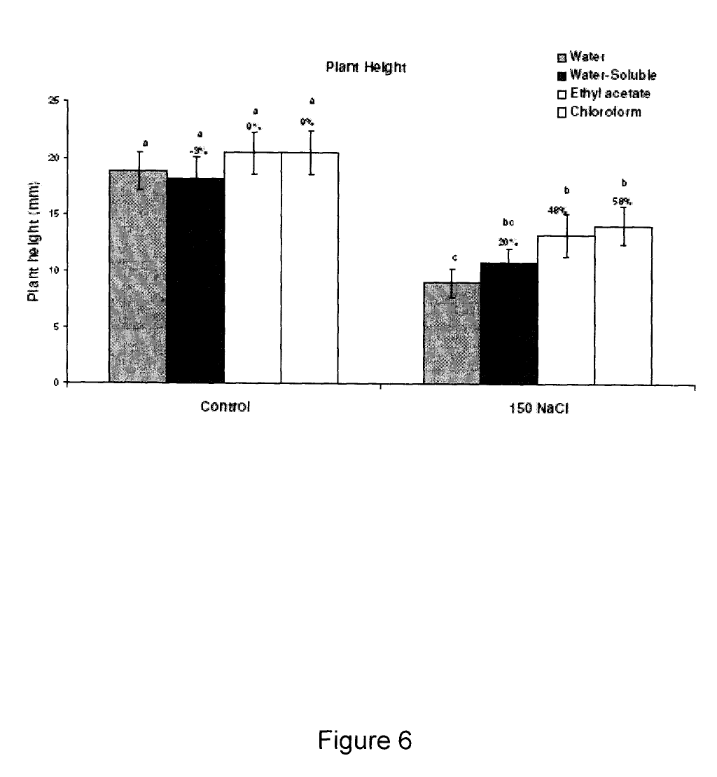

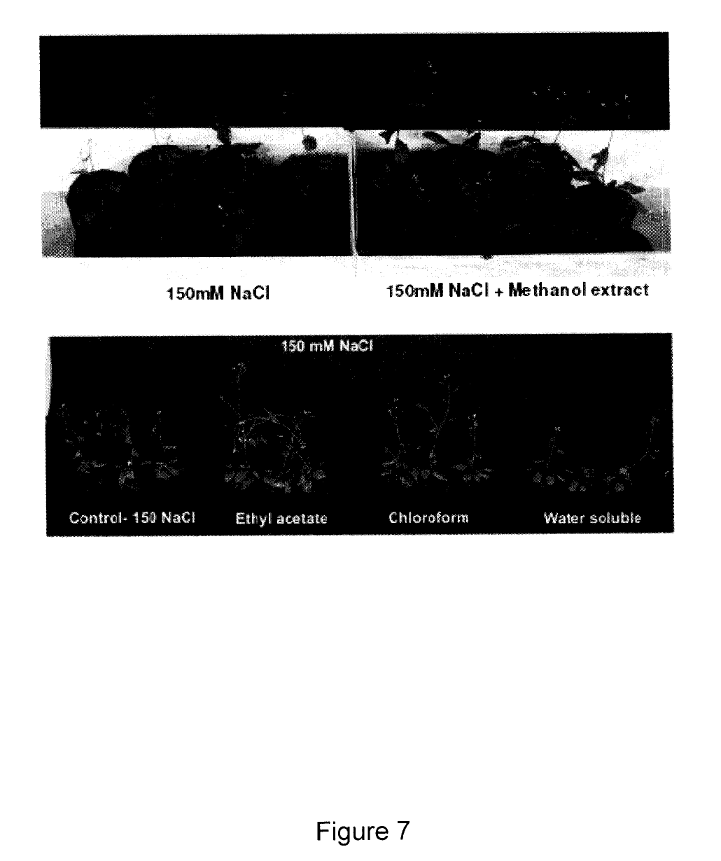

An analysis of plant fresh weight showed that under unstressed condition the extracts (water soluble, ethyl acetate and chloroform) did not affect the growth of A. thaliana plant. However, at 150 mM NaCl stress, ethyl acetate extract of A. nodosum showed 52% more fresh weight than plants treated with NaCl alone. Similarly, chloroform extract and water soluble fraction showed 36% and 13% more fresh weight over the untreated controls, respectively (FIG. 3). There was a significant increase in the number of leaves in the plants treated with different organic extracts of the algae. Ethyl acetate extract treated plants had 45% more leaves over the untreated control, while chloroform and water soluble also showed 15% and 11% increase (FIG. 4). Significant increase in leaf area was also observed in extract treated plants as compared to non treated plants, a 62% increase was observed in ethyl acetate extract treated plants (FIG. 5). Similar result was observed in regards to plant height (FIG. 6). FIG. 7 shows the A. thaliana plants following testing with the organic extracts of A. nodosum, clearly illustrating in pictorial view the salinity stress response seen in the graphical results of FIGS. 3-6.

Organic Compounds of A. nodosum Affect Gene Expression of Plant Resulting in Enhanced Tolerance to Salt-Induced Stress

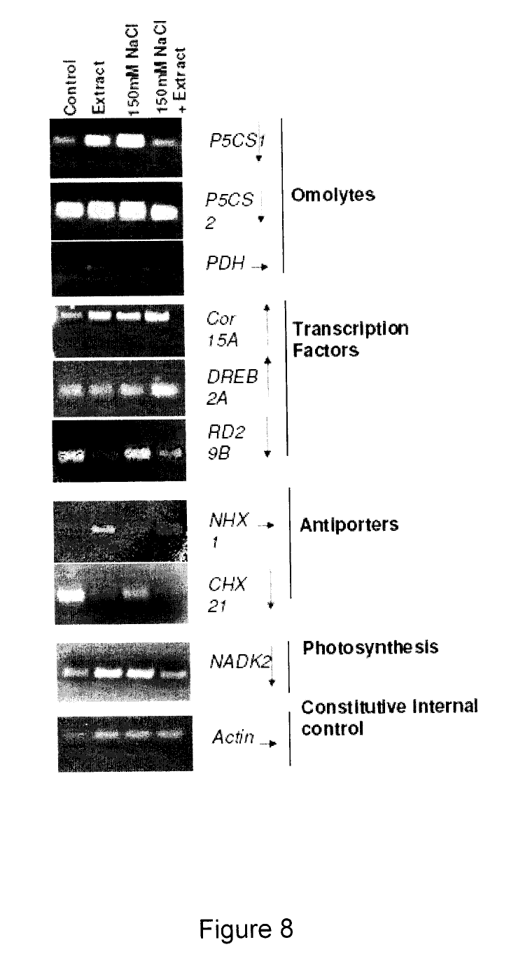

The expression of the following genes was studied by RT-PCR: P5CS1, P5CS2, PDH, Cor 15A, RD29B, DREB2A, NHX1, CHX21, NADK2. The overall pattern of A. nodosum induced gene expression is depicted in FIG. 8.

Salt stress (150 mM) induced proline synthetase genes P5CS1 and P5CS2, and addition of organic extracts of A. nodosum caused a reduction in P5CS1 and P5CS2 transcripts supporting our earlier observation of reduced proline content in the treated plants. No changes were observed in proline degrading gene, PDH due to extract treatment. Transcription factor Cor 15A was induced by extracts while it down regulated RD 29B expression.

Chemical Components of A. nodosum Reduces Na+ Uptake in Arabidopsis

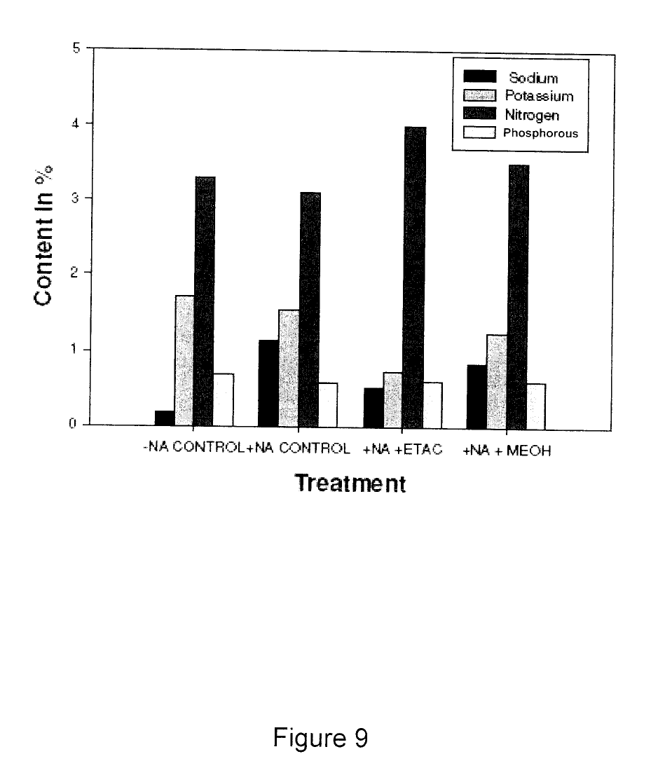

Plants exposed to high concentration of NaCl accumulate high concentration of Na+ in the tissue that leads to disruption of ionic balance and ultimately cellular function. We conducted an experiment to test if the methanol and ethyl acetate subfraction of A. nodosum affect the concentration of Na+ in the leaf tissue of Arabidopsis plants. Plant was treated with organic subfractions after 24 h exposure to 150 mM NaCl. As expected, sodium content in leaves of NaCl treated plants (150 mM) increased by 84% in comparison to control plants that were not root irrigated with NaCl. Interestingly, treatment of plants with ethyl acetate and methanol fractions caused a decreased accumulation of Na+ in the leaves. Ethyl acetate subfraction was the most active, it reduced the concentration of Na+ in the leaf tissue by 53% while methanol subfraction caused a reduction of 25%. Moreover, ethyl acetate and methanol fraction treatments also decreased potassium content by 56% and 26% respectively. On the other hand nitrogen and phosphorous content differed only slightly between untreated and treated controls (FIG. 9).

Organic Compounds of A. nodosum Elicit Global Transcriptome Changes in Arabidopsis Leaves

To study the molecular mechanisms of A. nodosum-elicited salt tolerance in Arabidopsis, we performed global gene expression profiling on the ATH1 GeneChip platform. The ATH1 GeneChip consists of over 22500-probe sets representing nearly 90% of the Arabidopsis genome, thus providing a means to ascertain global transcriptional changes elicited by organic sub-fractions of A. nodosum. Arabidopsis was exposed to 150 NaCl for 24 hours, after which treatment consisted of methanol or ethyl acetate sub-fraction of A. nodosum.

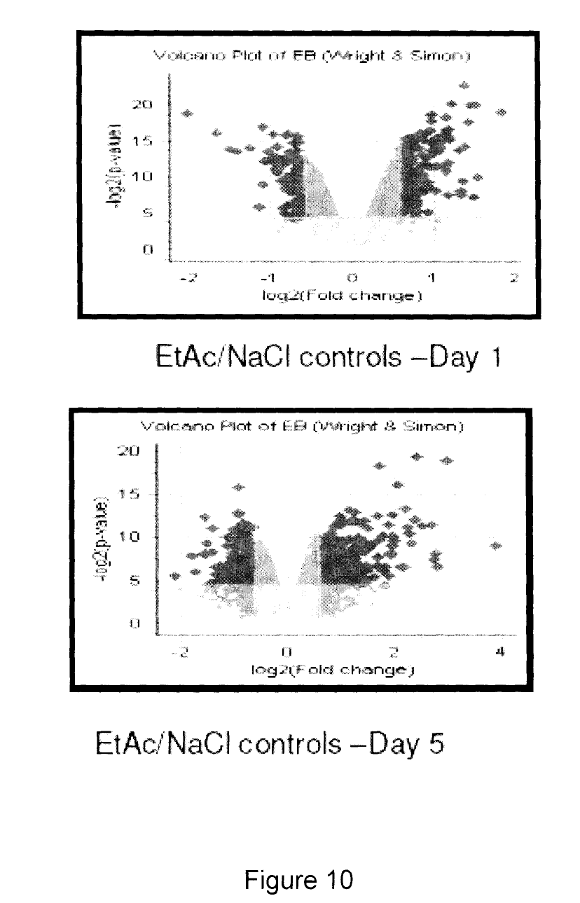

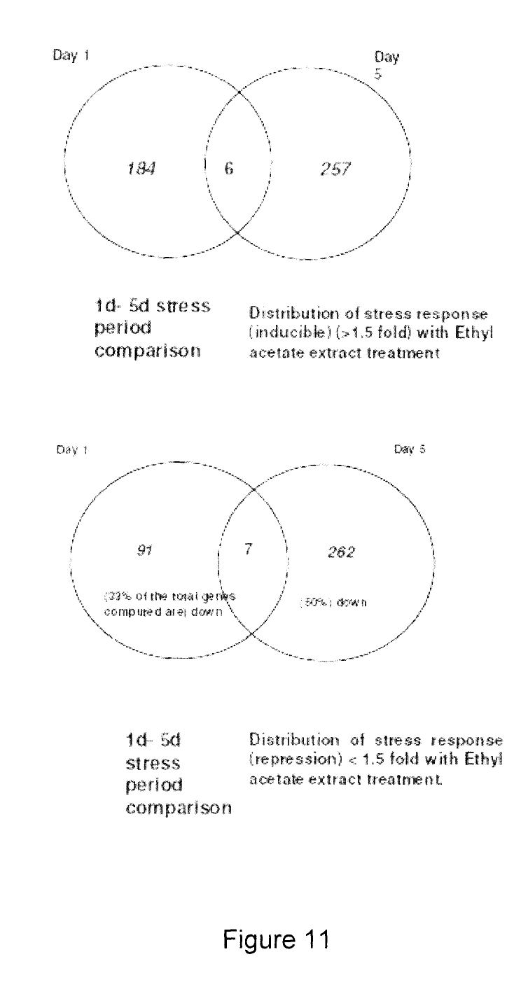

Global Expression profile: The change in global transcriptome elicited by organic components in A. nodosum extract is depicted in FIG. 10. Organic sub-fraction of A. nodosum caused significant changes in the transcription of a small subset of the genome, although the majority of transcripts remain unchanged by the treatment. There was little difference in the gene expression profile between the replicates within a treatment and a low p value and therefore, we used 1.5 fold level change as the cut off in our analysis. In the ethyl acetate sub-fraction treatment, 184 and 257 genes were up-regulated in day 1 and day 5, respectively. Only six genes were common in day 1 and 5. On the other hand, 91 and 262 genes were down-regulated on day 1 and day 5 by this treatment as illustrated in Venn diagrams (FIG. 11). Annotations were done based on MIPS Functional category classifications and listed (Table 1 -4).

Category 1: Up-Regulated Genes in Ethyl Actetate Subfraction Treatment on Day 1

Table 1 lists the genes that were up-regulated on day 1 of ethyl acetate sub-fraction treatment under 150 mM NaCl stress. Of the 184 genes that showed changes, the largest groups were annotated as involved in metabolism (27%), 16% were predicted to be involved in regulating gene expression i.e., transcription factors, 2.2% functions in abiotic stress response and 7.2 in cellular defense. Among all of gene responses, the transcript for late embryogenesis abundant 3 family protein/LEA3 family protein (AT1G02820) and myb-related transcription factor (CCA1; AT2G46830) was observed as the most strongly induced (2.731992 for LEA3; and 3.5 for CCA1). In the abiotic stress group, the genes that showed differential expression included: late embryogenesis abundant protein LEA group 1 (AT5g06760) and LEA 3 family (AT1G02820); drought-responsive protein (AT4g15910) and HVA 22d genes (AT4g24960). The synthesis of hydrophilic proteins is a major response to water-deficit conditions like salinity and drought. LEA proteins, first characterized in cotton during the late stages of seed embryogenesis are a group of hydrophilic proteins and the encoding genes are ABA inducible. Previous studies report that LEA group 1 (AT5g06760) is regulated by ABA (Zalejski et al., 2006). LEA proteins play a protective role in the dry state and contribute to desiccation tolerance (reviewed by Oliver and Bewley, 1997; Kermode, 1997), although details of their mode of action are not yet clear. However, recent in vitro studies suggest that group I and group III LEA help prevent protein aggregation (Goyal et al., 2005). Also included in the abiotic stress category are other ABA inducible genes: HVA22d (AT4g24960) and Di21 (AT4g15910).

TABLE 1
Microarray data for selected genes induced by salinity stress in Arabidopsis thaliana by
ethyl acetate extract treatment after Day 1 of treatment

| | | | |
|---|---|---|---|
| Array Element | Locus Identifier | Fold (log₂) | Annotation |

Abiotic stress

| | | | |
|---|---|---|---|
| 262113_at | AT1G02820 | 2.731992 | late embryogenesis abundant 3 family protein/ |
| | | | LEA3 family protein |
| 245523_at | AT4G15910 | 1.664332 | drought-responsive protein/drought-induced |
| | | | protein (Di21) |
| 250648_at | AT5G06760 | 1.560875 | late embryogenesis abundant group 1 domain- |
| | | | containing protein/LEA group 1 domain- |
| | | | containing protein |
| 254085_at | AT4G24960 | 1.542183 | ABA-responsive protein (HVA22d) |

Cellular organization and biogenesis

| | | | |
|---|---|---|---|
| 252607_at | AT3G44990 | 2.938349 | xyloglucan:xyloglucosyl transferase, putative/ |
| | | | xyloglucan endotransglycosylase, putative/ |
| | | | endo-xyloglucan transferase, putative |
| 245465_at | AT4G16590 | 2.862751 | glucosyltransferase-related |
| 259736_at | AT1G64390 | 1.546902 | endo-1,4-beta-glucanase, putative/cellulase, |
| | | | putative |
| 258675_at | AT3G08770 | 2.547661 | lipid transfer protein 6 (LTP6) |
| 253815_at | AT4G28250 | 1.841339 | beta-expansin, putative (EXPB3) |
| 256924_at | AT3G29590 | 1.739235 | transferase family protein |
| | | | AT5MAT; O-malonyltransferase/transferase |
| 254185_at | AT4G23990 | 1.709051 | cellulose synthase family protein |
| | | | ATCSLG3 (Cellulose synthase-like G3); |
| | | | transferase/transferase, transferring glycosyl |
| | | | groups |
| 254773_at | AT4G13410 | 1.653896 | glycosyl transferase family 2 protein |
| 255524_at | AT4G02330 | 1.583472 | pectinesterase family protein |
| 267115_s_at | AT2G32540 | 1.516453 | cellulose synthase family protein /// cellulose |
| | AT2G32530 | | synthase family protein |
| | | | [AT2G32540, ATCSLB04 (Cellulose synthase- |
| | | | like B4); transferase/transferase, transferring |
| | | | glycosyl groups]; [AT2G32530, ATCSLB03 |
| | | | (Cellulose synthase-like B3); transferase/ |
| | | | transferase, transferring glycosyl groups] |
| 256243_at | AT3G12500 | 1.529772 | basic endochitinase |
| | | | ATHCHIB (BASIC CHITINASE); chitinase |

Unknown genes

| | | | |
|---|---|---|---|
| 265892_at | AT2G15020 | 2.609756 | expressed protein |
| | | | similar to unknown protein [Arabidopsis |
| | | | thaliana] (TAIR: AT5G64190.1); similar to |
| | | | hypothetical protein [Oryza sativa (japonica |
| | | | cultivar-group)] (GB: AAP46203.1) |
| 256266_at | AT3G12320 | 2.335075 | expressed protein |
| | | | similar to unknown protein [Arabidopsis |
| | | | thaliana] (TAIR: AT5G06980.1); similar to |
| | | | ACI112 [Lycopersicon esculentum] |
| | | | (GB: AAY97870.1) |
| 250292_at | AT5G13220 | 2.280981 | expressed protein |
| | | | similar to unknown protein [Arabidopsis |
| | | | thaliana] (TAIR: AT5G20900.1); similar to |
| | | | P0482D04.10 [Oryza sativa (japonica cultivar- |
| | | | group)] (GB: BAB89663.1); similar to |
| | | | Os04g0395800 [Oryza sativa (japonica cultivar- |
| | | | group)] (GB: NP_001052661.1); similar to |
| | | | Os10g0392400 [Oryza sativa (japonica cultivar- |
| | | | group)] (GB: NP_001064513.1); contains |
| | | | InterPro domain ZIM; (InterPro: IPR010399) |
| 250665_at | AT5G06980 | 1.977186 | expressed protein |
| | | | similar to unknown protein [Arabidopsis |
| | | | thaliana] (TAIR: AT3G12320.1) |
| 251727_at | AT3G56290 | 1.897128 | expressed protein |
| | | | similar to Os01g0823600 [Oryza sativa |
| | | | (japonica cultivar-group)] |
| | | | (GB: NP_001044661.1); similar to unnamed |
| | | | protein product [Ostreococcus tauri] |
| | | | (GB: CAL58546.1) |
| 247814_at | AT5G58310 | 1.818424 | hydrolase, alpha/beta fold family protein |
| | | | hydrolase, alpha/beta fold family protein |
| 251869_at | AT3G54500 | 1.806031 | expressed protein |
| | | | similar to dentin sialophosphoprotein-related |
| | | | [Arabidopsis thaliana] (TAIR: AT5G64170.2); |
| | | | similar to conserved hypothetical protein |
| | | | [Medicago truncatula] (GB: ABD28297.1) |
| 256096_at | AT1G13650 | 1.766908 | expressed protein |
| | | | similar to 18S pre-ribosomal assembly protein |
| | | | gar2-related [Arabidopsis thaliana] |
| | | | (TAIR: AT2G03810.3); similar to hypothetical |
| | | | protein [Trypanosoma cruzi strain CL Brener] |
| | | | (GB: XP_813437.1) |
| 262452_at | AT1G11210 | 1.727922 | expressed protein |
| | | | similar to unknown protein [Arabidopsis |
| | | | thaliana] (TAIR: AT1G11220.1); similar to fiber |
| | | | expressed protein [Gossypium hirsutum] |
| | | | (GB: AAY85179.1); similar to cotton fiber |
| | | | expressed protein 1 [Gossypium hirsutum] |
| | | | (GB: AAC33276.1); contains InterPro domain |
| | | | Protein of unknown function DUF761, plant; |
| | | | (InterPro: IPR008480) |
| 248205_at | AT5G54300 | 1.712183 | expressed protein |
| | | | similar to unknown protein [Arabidopsis |
| | | | thaliana] (TAIR: AT1G61260.1); similar to |
| | | | Protein of unknown function DUF761, plant |
| | | | [Medicago truncatula] (GB: ABE84235.1); |
| | | | contains InterPro domain Protein of unknown |
| | | | function DUF761, plant; (InterPro: IPR008480) |
| 247030_at | AT5G67210 | 1.707944 | expressed protein |
| | | | nucleic acid binding/pancreatic ribonuclease |
| 247463_at | AT5G62210 | 1.707315 | embryo-specific protein-related |
| 247214_at | AT5G64850 | 1.698314 | expressed protein |
| | | | similar to unknown protein [Arabidopsis |
| | | | thaliana] (TAIR: AT5G09960.1); similar to |
| | | | 80C09_15 [Brassica rapa subsp. pekinensis] |
| | | | (GB: AAZ41826.1) |
| 249932_at | AT5G22390 | 1.689324 | expressed protein |
| | | | similar to unknown protein [Arabidopsis |
| | | | thaliana] (TAIR: AT5G19260.1); similar to |
| | | | hypothetical protein [Oryza sativa (japonica |
| | | | cultivar-group)] (GB: BAD28434.1); similar to |
| | | | Os12g0155100 [Oryza sativa (japonica cultivar- |
| | | | group)] (GB: NP_001066192.1) |
| 264636_at | AT1G65490 | 1.683121 | expressed protein |
| | | | similar to unknown protein [Arabidopsis |
| | | | thaliana] (TAIR: AT1G65500.1) |
| 252661_at | AT3G44450 | 1.670119 | expressed protein |
| | | | similar to unknown protein [Arabidopsis |
| | | | thaliana] (TAIR: AT3G52740.1); similar to |
| | | | H0717B12.8 [Oryza sativa (indica cultivar- |
| | | | group)] (GB: CAH67161.1) |
| 250158_at | AT5G15190 | 1.617973 | expressed protein |
| | | | unknown protein |
| 266800_at | AT2G22880 | 1.60535 | VQ motif-containing protein |
| 261033_at | AT1G17380 | 1.60292 | expressed protein |
| | | | similar to unknown protein [Arabidopsis |
| | | | thaliana] (TAIR: AT1G72450.1); similar to |
| | | | PnFL-2 [Ipomoea nil] (GB: AAG49896.1); |
| | | | similar to Os07g0615200 [Oryza sativa |
| | | | (japonica cultivar-group)] |
| | | | (GB: NP_001060268.1); similar to |
| | | | Os03g0402800 [Oryza sativa (japonica cultivar- |
| | | | group)] (GB: NP_001050322.1); contains |
| | | | InterPro domain ZIM; (InterPro: IPR010399) |
| 261247_at | AT1G20070 | 1.602362 | expressed protein |
| 260804_at | AT1G78410 | 1.589149 | VQ motif-containing protein |
| 247933_at | AT5G56980 | 1.569768 | expressed protein |
| | | | similar to unknown protein [Arabidopsis |
| | | | thaliana] (TAIR: AT4G26130.1); similar to |
| | | | cDNA-5-encoded protein (GB: AAA50235.1) |
| 253165_at | AT4G35320 | 1.569475 | expressed protein |
| | | | similar to unknown protein [Arabidopsis |
| | | | thaliana] (TAIR: AT2G17300.1); similar to |
| | | | Os02g0715300 [Oryza sativa (japonica cultivar- |
| | | | group)] (GB: NP_001047925.1); similar to |
| | | | Os08g0511400 [Oryza sativa (japonica cultivar- |
| | | | group)] (GB: NP_001062213.1); contains |
| | | | domain N-terminal domain of cbl (N-cbl) |
| | | | (SSF47668) |
| 255763_at | AT1G16730 | 1.562988 | expressed protein |
| 263796_at | AT2G24540 | 1.561418 | kelch repeat-containing F-box family protein |
| | AT2G24545 | | |
| 246125_at | AT5G19875 | 1.558457 | expressed protein |
| | | | similar to oxidoreductase/transition metal ion |
| | | | binding [Arabidopsis thaliana] |
| | | | (TAIR: AT2G31940.1); similar to conserved |
| | | | hypothetical protein [Medicago truncatula] |
| | | | (GB: ABE77569.1) |
| 253943_at | AT4G27030 | 1.555481 | expressed protein |
| | | | similar to unknown protein [Arabidopsis |
| | | | thaliana] (TAIR: AT1G62190.1); similar to |
| | | | Os08g0187900 [Oryza sativa (japonica cultivar- |
| | | | group)] (GB: NP_001061153.1); similar to |
| | | | Ubiquitin-conjugating enzyme (ISS) |
| | | | [Ostreococcus tauri] (GB: CAL55480.1); |
| | | | contains domain UBIQUITIN-CONJUGATING |
| | | | ENZYME VARIANT 1 (PTHR11621: SF1); |
| | | | contains domain UBIQUITIN-CONJUGATING |
| | | | ENZYME E2 (PTHR11621) |
| 249191_at | AT5G42760 | 1.542773 | O-methyltransferase N-terminus domain- |
| | | | containing protein |
| | | | similar to hypothetical protein [Oryza sativa |
| | | | (japonica cultivar-group)] (GB: AAN65019.1); |
| | | | contains InterPro domain O-methyltransferase, |
| | | | N-terminal; (InterPro: IPR003455); contains |
| | | | InterPro domain Protein of unknown function |
| | | | Mtu_121; (InterPro: IPR011610) |
| 257710_at | AT3G27350 | 1.522115 | expressed protein |
| | | | similar to unknown protein [Arabidopsis |
| | | | thaliana] (TAIR: AT5G40700.1); similar to |
| | | | Targeting for Xklp2 [Medicago truncatula] |
| | | | (GB: ABE84619.1) |
| 249918_at | AT5G19240 | 1.520507 | expressed protein |
| | | | Identical to Putative GPI-anchored protein |
| | | | At5g19240 precursor [Arabidopsis Thaliana] |
| | | | (GB: Q84VZ5; GB: Q8H7A4); similar to |
| | | | unknown protein [Arabidopsis thaliana] |
| | | | (TAIR: AT5G19230.1); similar to |
| | | | Os07g0645000 [Oryza sativa (japonica cultivar- |
| | | | group)] (GB: NP_001060451.1); similar to |
| | | | unknown protein [Oryza sativa (japonica |
| | | | cultivar-group)] (GB: BAC07018.1); contains |
| | | | domain PR-1-like (SSF55797) |
| 249134_at | AT5G43150 | 1.517847 | expressed protein |
| | | | similar to hypothetical protein |
| | | | MtrDRAFT_AC141109g4v1 [Medicago |
| | | | truncatula] (GB: ABE79896.1) |
| 249011_at | AT5G44670 | 1.514902 | expressed protein |
| | | | similar to unknown protein [Arabidopsis |
| | | | thaliana] (TAIR: AT4G20170.1); similar to |
| | | | Os06g0328800 [Oryza sativa (japonica cultivar- |
| | | | group)] (GB: NP_001057533.1); similar to |
| | | | Os02g0712500 [Oryza sativa (japonica cultivar- |
| | | | group)] (GB: NP_001047907.1); similar to |
| | | | unknown protein [Oryza sativa (japonica |
| | | | cultivar-group)] (GB: BAD72474.1); contains |
| | | | InterPro domain Protein of unknown function |
| | | | DUF23; (InterPro: IPR008166) |
| 254508_at | AT4G20170 | 1.512831 | expressed protein |
| | | | similar to unknown protein [Arabidopsis |
| | | | thaliana] (TAIR: AT5G44670.1); similar to |
| | | | Os06g0328800 [Oryza sativa (japonica cultivar- |
| | | | group)] (GB: NP_001057533.1); similar to |
| | | | Os02g0712500 [Oryza sativa (japonica cultivar- |
| | | | group)] (GB: NP_001047907.1); similar to |
| | | | unknown protein [Oryza sativa (japonica |
| | | | cultivar-group)] (GB: BAD72474.1); contains |
| | | | InterPro domain Protein of unknown function |
| | | | DUF23; (InterPro: IPR008166) |
| 253425_at | AT4G32190 | 1.512381 | centromeric protein-related |
| 259927_at | AT1G75100 | 1.506455 | expressed protein |
| | | | JAC1 (J-DOMAIN PROTEIN REQUIRED |
| | | | FOR CHLOROPLAST ACCUMULATION |
| | | | RESPONSE 1); heat shock protein binding |
| 253891_at | | 1.500037 | expressed protein |
| | | | similar to unknown protein [Arabidopsis |
| | | | thaliana] (TAIR: AT1G64650.1); similar to |
| | | | unknown protein [Arabidopsis thaliana] |
| | | | (TAIR: AT3G49310.1); similar to expressed |
| | | | protein [Oryza sativa (japonica cultivar-group)] |
| | | | (GB: ABF93637.1); similar to Os10g0519600 |
| | | | [Oryza sativa (japonica cultivar-group)] |
| | | | (GB: NP_001065080.1); similar to Major |
| | | | Facilitator Superfamily protein, expressed |
| | | | [Oryza sativa (japonica cultivar-group)] |
| | | | (GB: ABB47893.2); contains InterPro domain |
| | | | Protein of unknown function DUF791; |
| | | | (InterPro: IPR008509) |

Metabolism

| | | | |
|---|---|---|---|
| 264511_at | AT1G09350 | 2.690889 | galactinol synthase, putative |
| | | | ATGOLS3 (ARABIDOPSIS THALIANA |
| | | | GALACTINOL SYNTHASE 3); transferase, |
| | | | transferring glycosyl groups/transferase, |
| | | | transferring hexosyl groups |
| 261191_at | AT1G32900 | 2.313856 | starch synthase, putative |
| 266532_at | AT2G16890 | 2.057935 | UDP-glucoronosyl/UDP-glucosyl transferase |
| | | | family protein |
| 260727_at | AT1G48100 | 2.017334 | glycoside hydrolase family 28 protein/ |
| | | | polygalacturonase (pectinase) family protein |
| 252011_{—} | AT3G52720.1 | 2.033333 | carbonic anhydrase family |
| | | | protein |
| 249411_at | AT5G40390 | 1.945845 | raffinose synthase family protein |
| 251984_at | AT3G53260 | 1.789189 | phenylalanine ammonia-lyase 2 (PAL2) |
| 263845_at | AT2G37040 | 1.55923 | phenylalanine ammonia-lyase 1 (PAL1) |
| 258589_at | AT3G04290 | 1.752056 | GDSL-motif lipase/hydrolase family protein |
| | | | ATLTL1/LTL1 (LI-TOLERANT LIPASE 1); |
| | | | carboxylic ester hydrolase |
| 263841_at | AT2G36870 | 1.725838 | xyloglucan:xyloglucosyl transferase, putative/ |
| | | | xyloglucan endotransglycosylase, putative/ |
| | | | endo-xyloglucan transferase, putative |
| 247360_at | AT5G63450 | 2.286131 | cytochrome P450, putative |
| | | | CYP94B1 (cytochrome P450, family 94, |
| | | | subfamily B, polypeptide 1); oxygen binding |
| 252911_at | AT4G39510 | 1.944384 | cytochrome P450 family protein |
| | | | CYP96A12 (cytochrome P450, family 96, |
| | | | subfamily A, polypeptide 12); oxygen binding |
| 266246_at | AT2G27690 | 1.821355 | cytochrome P450, putative |
| | | | CYP94C1 (cytochrome P450, family 94, |
| | | | subfamily C, polypeptide 1); oxygen binding |
| 249215_at | AT5G42800 | 2.829937 | dihydroflavonol 4-reductase (dihydrokaempferol |
| | | | 4-reductase) (DFR) |
| 245624_at | AT4G14090 | 1.7226 | UDP-glucoronosyl/UDP-glucosyl transferase |
| | | | family protein |
| 260955_at | AT1G06000 | 1.712522 | UDP-glucoronosyl/UDP-glucosyl transferase |
| | | | family protein |
| 250794_at | AT5G05270 | 2.499514 | chalcone-flavanone isomerase family protein |
| 250207_at | AT5G13930 | 2.229782 | chalcone synthase/naringenin-chalcone |
| | | | synthase |
| 252123_at | AT3G51240 | 2.095258 | naringenin 3-dioxygenase/flavanone 3- |
| | | | hydroxylase (F3H) |
| | | | F3H (TRANSPARENT TESTA 6); naringenin |
| | | | 3-dioxygenase |
| 250533_at | AT5G08640 | 1.989595 | flavonol synthase 1 (FLS1) |
| 249774_at | AT5G24150 | 1.676203 | squalene monooxygenase 1, 1/squalene |
| | | | epoxidase 1, 1 (SQP1,1) |
| 266778_at | AT2G29090 | 1.651743 | cytochrome P450 family protein |
| 259681_at | AT1G77760 | 1.566168 | nitrate reductase 1 (NR1) |
| 251827_at | AT3G55120 | 1.616842 | chalcone-flavanone isomerase/chalcone |
| | | | isomerase (CHI) |
| | | | TT5 (TRANSPARENT TESTA 5); chalcone |
| | | | isomerase |
| 258613_at | AT3G02870 | 1.581021 | inositol-1(or 4)-monophosphatase, putative/ |
| | | | inositol monophosphatase, putative/IMPase, |
| | | | putative |
| | | | VTC4; 3′(2′),5′-bisphosphate nucleotidase/ |
| | | | inositol or phosphatidylinositol phosphatase |
| 254662_at | AT4G18270 | 1.615176 | glycosyl transferase family 4 protein |
| | | | ATTRANS11 (Arabidopsis thaliana translocase |
| | | | 11); catalytic |
| 252363_at | AT3G48460 | 1.61335 | GDSL-motif lipase/hydrolase family protein |
| 245275_at | AT4G15210 | 1.609439 | beta-amylase (BMY1)/1,4-alpha-D-glucan |
| | | | maltohydrolase |
| 248311_at | AT5G52570 | 1.607389 | beta-carotene hydroxylase, putative |
| | | | BETA-OHASE 2 (BETA-CAROTENE |
| | | | HYDROXYLASE 2); beta-carotene |
| | | | hydroxylase |
| 245734_at | AT1G73480 | 1.59682 | hydrolase, alpha/beta fold family protein |
| 250832_at | AT5G04950 | 1.587492 | nicotianamine synthase, putative |
| 250344_at | AT5G11930 | 1.583021 | glutaredoxin family protein |
| 264931_at | AT1G60590 | 1.572077 | polygalacturonase, putative/pectinase, putative |
| 256057_at | AT1G07180 | 1.570594 | pyridine nucleotide-disulphide oxidoreductase |
| | | | family protein |
| | | | NDA1 (ALTERNATIVE NAD(P)H |
| | | | DEHYDROGENASE 1); NADH dehydrogenase |
| 263433_at | AT2G22240 | 1.553779 | inositol-3-phosphate synthase isozyme 2/myo- |
| | | | inositol-1-phosphate synthase 2/MI-1-P |
| | | | synthase 2/IPS 2 |
| 259445_at | AT1G02400 | 1.538629 | gibberellin 2-oxidase, putative/GA2-oxidase, |
| | | | putative |
| | | | ATGA2OX6/DTA1 (GIBBERELLIN 2- |
| | | | OXIDASE 6); gibberellin 2-beta-dioxygenase |
| 246700_at | AT5G28030 | 1.538054 | cysteine synthase, putative/O-acetylserine |
| | | | (thiol)-lyase, putative/O-acetylserine |
| | | | sulfhydrylase, putative |
| 262039_at | AT1G80050 | 1.537622 | adenine phosphoribosyltransferase 2 (APT2) |
| 261907_at | AT1G65060 | 1.535029 | 4-coumarate--CoA ligase 3/4-coumaroyl-CoA |
| | | | synthase 3 (4CL3) |
| 267429_at | AT2G34850 | 1.533589 | NAD-dependent epimerase/dehydratase family |
| | | | protein |
| | | | MEE25 (maternal effect embryo arrest 25); |
| | | | catalytic |
| 266391_at | AT2G41290 | 1.532358 | strictosidine synthase family protein |
| 249540_at | AT5G38120 | 1.530133 | 4-coumarate--CoA ligase family protein/4- |
| | | | coumaroyl-CoA synthase family protein |
| 245627_at | AT1G56600 | 1.518096 | galactinol synthase, putative |
| | | | ATGOLS2 (ARABIDOPSIS THALIANA |
| | | | GALACTINOL SYNTHASE 2); transferase, |
| | | | transferring glycosyl groups/transferase, |
| | | | transferring hexosyl groups |
| 247175_at | AT5G65280 | 1.516934 | lanthionine synthetase C-like family protein |
| 261914_at | AT1G65870 | 1.514853 | disease resistance-responsive family protein |
| 261046_at | AT1G01390 | 1.513808 | UDP-glucoronosyl/UDP-glucosyl transferase |
| | | | family protein |
| 246490_at | AT5G15950.1 | 2.141977 | adenosylmethionine decarboxylase family |
| | | | protein |
| 246468_at | AT5G17050 | 1.902498 | UDP-glucoronosyl/UDP-glucosyl transferase |
| | | | family protein |

Other genes

| | | | |
|---|---|---|---|
| 256020_at | AT1G58290 | 1.528062 | glutamyl-tRNA reductase 1/GluTR (HEMA1) |
| 264758_at | AT1G61340 | 2.169349 | F-box family protein |
| 265962_at | AT2G37460 | 1.815725 | nodulin MtN21 family protein |
| 248467_at | AT5G50800 | 1.737688 | nodulin MtN3 family protein |
| 264217_at | AT1G60190 | 1.70939 | armadillo/beta-catenin repeat family protein/U- |
| | | | box domain-containing protein |
| 249798_at | AT5G23730 | 1.556476 | transducin family protein/WD-40 repeat family |
| | | | protein |
| | | | nucleotide binding |
| 248961_at | AT5G45650 | 1.535671 | subtilase family protein |
| 266993_at | AT2G39210 | 1.53321 | nodulin family protein |
| 248676_at | AT5G48850 | 1.517959 | male sterility MS5 family protein |
| 253696_at | AT4G29740 | 1.824141 | FAD-binding domain-containing protein/ |
| | | | cytokinin oxidase family protein |
| 256894_at | AT3G21870 | 1.574575 | cyclin family protein |
| | | | CYCP2; 1 (cyclin p2; 1); cyclin-dependent |
| | | | protein kinase |

Signal transduction

| | | | |
|---|---|---|---|
| 264767_at | AT1G61380 | 1.782612 | S-locus protein kinase, putative |
| 248910_at | AT5G45820 | 1.737011 | CBL-interacting protein kinase 20 (CIPK20) |
| 248607_at | AT5G49480 | 1.691496 | sodium-inducible calcium-binding protein |
| | | | (ACP1)/ |
| | | | sodium-responsive calcium-binding protein |
| | | | (ACP1) |
| 260774_at | AT1G78290 | 1.66375 | serine/threonine protein kinase, putative |
| 251054_at | AT5G01540 | 1.603513 | lectin protein kinase, putative |
| 248191_at | AT5G54130 | 1.545963 | calcium-binding EF hand family protein |
| 265030_at | AT1G61610 | 1.533198 | S-locus lectin protein kinase family protein |
| 261089_at | AT1G07570 | 1.506941 | protein kinase (APK1a) |
| 267546_at | AT2G32680 | 1.502409 | disease resistance family protein |
| 258119_at | AT3G14720 | 1.500632 | mitogen-activated protein kinase, putative/ |
| | | | MAPK, putative (MPK19) |

TRANSCRIPTION FACTORS

| | | | |
|---|---|---|---|
| 266719_at | AT2G46830 | 3.590629 | myb-related transcription factor (CCA1) |
| 261569_at | AT1G01060 | 2.253258 | myb family transcription factor |
| 263739_at | AT2G21320 | 2.252043 | zinc finger (B-box type) family protein |
| 266720_s_at | AT2G46670 | 2.214092 | pseudo-response regulator, putative/timing of |
| | | | CAB expression 1-like protein, putative /// |
| | | | pseudo-response regulator, putative/timing of |
| | | | CAB expression 1-like protein, putative |
| 257262_at | AT3G21890 | 2.077132 | zinc finger (B-box type) family protein |
| 253799_at | AT4G28140 | 2.064143 | AP2 domain-containing transcription factor, |
| | | | putative |
| 263252_at | AT2G31380 | 1.991303 | zinc finger (B-box type) family protein/salt |
| | | | tolerance-like protein (STH) |
| 254066_at | AT4G25480 | 1.963332 | DRE-binding protein (DREB1A)/CRT/DRE- |
| | | | binding factor 3 (CBF3) |
| 250099_at | AT5G17300 | 1.902815 | myb family transcription factor |
| 249769_at | AT5G24120 | 1.860477 | RNA polymerase sigma subunit SigE (sigE)/ |
| | | | sigma-like factor (SIG5) |
| 254075_at | AT4G25470 | 1.845192 | DRE-binding protein (DREB1C)/CRT/DRE- |
| | | | binding factor 2 (CBF2) |
| 259466_at | AT1G19050 | 1.814394 | two-component responsive regulator/response |
| | | | regulator 7 (ARR7) |
| 258497_at | AT3G02380 | 1.772366 | zinc finger protein CONSTANS-LIKE 2 |
| | | | (COL2) |
| 266820_at | AT2G44940 | 1.718757 | AP2 domain-containing transcription factor |
| | | | TINY, putative |
| 256185_at | AT1G51700 | 1.676723 | Dof-type zinc finger domain-containing protein |
| | | | (ADOF1) |
| 251665_at | AT3G57040 | 1.663682 | two-component responsive regulator/response |
| | | | reactor 4 (RR4) |
| 265418_at | AT2G20880 | 1.625219 | AP2 domain-containing transcription factor, |
| | | | putative |
| 255016_at | AT4G10120 | 1.624597 | sucrose-phosphate synthase, putative |
| 252917_at | AT4G38960 | 1.610185 | zinc finger (B-box type) family protein |
| 245078_at | AT2G23340 | 1.593763 | AP2 domain-containing transcription factor, |
| | | | putative |
| 263664_at | AT1G04250 | 1.591111 | auxin-responsive protein/indoleacetic acid- |
| | | | induced protein 17 (IAA17) |
| 264057_at | AT2G28550 | 1.589255 | AP2 domain-containing transcription factor |
| | | | RAP2.7 (RAP2.7) |
| 250598_at | AT5G07690 | 1.584711 | myb family transcription factor (MYB29) |
| 246523_at | AT5G15850 | 1.582355 | zinc finger protein CONSTANS-LIKE 1 |
| | | | (COL1) |
| 248246_at | AT5G53200 | 1.573483 | myb family transcription factor |
| | | | (TRIPTYCHON) |
| 261375_at | AT1G53160 | 1.570211 | squamosa promoter-binding protein-like 4 |
| | | | (SPL4) |
| 252214_at | AT3G50260 | 1.558081 | AP2 domain-containing transcription factor, |
| | | | putative |
| 264692_at | AT1G70000 | 1.543164 | DNA-binding family protein |
| 253411_at | AT4G32980 | 1.535375 | homeobox protein (ATH1) |

CELLULAR RESCUE AND DEFENSE

| | | | |
|---|---|---|---|
| 261037_at | AT1G17420 | 2.157759 | lipoxygenase, putative |
| 263384_at | AT2G40130 | 1.938878 | heat shock protein-related |
| 250083_at | AT5G17220 | 1.909543 | glutathione S-transferase, putative |
| 260399_at | AT1G72520 | 1.737015 | lipoxygenase, putative |
| 266992_at | AT2G39200 | 1.694233 | seven transmembrane MLO family protein/ |
| | | | MLO-like protein 12 (MLO12) |
| 253104_at | AT4G36010 | 1.655617 | pathogenesis-related thaumatin family protein |
| 258791_at | AT3G04720 | 1.63341 | hevein-like protein (HEL) |
| 252921_at | AT4G39030 | 1.623038 | enhanced disease susceptibility 5 (EDS5)/ |
| | | | salicylic acid induction deficient 1 (SID1) |
| 261150_at | AT1G19640 | 1.606685 | S-adenosyl-L-methionine:jasmonic acid |
| | | | carboxyl methyltransferase (JMT) |
| 257644_at | AT3G25780 | 1.58864 | allene oxide cyclase, putative/early-responsive |
| | | | to dehydration protein, putative/ERD protein, |
| | | | putative |
| 253496_at | AT4G31870 | 1.583689 | glutathione peroxidase, putative |
| 260831_at | AT1G06830 | 1.511424 | glutaredoxin family protein |
| 261958_at | AT1G64500 | 1.846779 | glutaredoxin family protein |

TRANSPORTERS

| | | | |
|---|---|---|---|
| 261881_at | AT1G80760 | 2.267071 | major intrinsic family protein/MIP family |
| | | | protein |
| 264400_at | AT1G61800 | 2.264749 | glucose-6-phosphate/phosphate translocator, |
| | | | putative |
| 256751_at | AT3G27170 | 2.069786 | chloride channel protein (CLC-b) |
| 262883_at | AT1G64780 | 2.039022 | ammonium transporter 1, member 2 (AMT1.2) |
| 252414_at | AT3G47420 | 2.003536 | glycerol-3-phosphate transporter, putative/ |
| | | | glycerol 3-phosphate permease, putative |
| 263918_at | AT2G36590 | 1.808529 | proline transporter, putative |
| 260676_at | AT1G19450 | 1.772287 | integral membrane protein, putative/sugar |
| | | | transporter family protein |
| 255877_at | AT2G40460 | 1.667473 | proton-dependent oligopeptide transport (POT) |
| | | | family protein |
| 249765_at | AT5G24030 | 1.659875 | C4-dicarboxylate transporter/malic acid |
| | | | transport family protein |
| 259185_at | AT3G01550 | 1.640112 | triose phosphate/phosphate translocator, putative |
| 262526_at | AT1G17050 | 1.632548 | geranyl diphosphate synthase, putative/GPPS, |
| | | | putative/dimethylallyltransferase, putative/ |
| | | | prenyl transferase, putative |
| 256336_at | AT1G72030 | 1.627288 | GCN5-related N-acetyltransferase (GNAT) |
| | | | family protein |
| 246310_at | AT3G51895 | 1.621192 | sulfate transporter (ST1) |
| 250926_at | AT5G03555 | 1.589828 | permease, cytosine/purines, uracil, thiamine, |
| | | | allantoin family protein |
| 258293_at | AT3G23430 | 1.535768 | phosphate transporter, putative (PHO1) |

Energy

| | | | |
|---|---|---|---|
| 265722_at | AT2G40100 | 1.716622 | chlorophyll A-B binding protein (LHCB4.3) |
| 245242_at | AT1G44446 | 1.630978 | chlorophyll a oxygenase (CAO)/chlorophyll b |
| | | | synthase |
| 258321_at | AT3G22840 | 2.604578 | chlorophyll A-B binding family protein/early |
| | | | light-induced protein (ELIP) |

Genes involved in cellular organization and biogenesis like lipid transfer family protein LTP6 (AT3g08770); endo-1,4-beta-glucanase, putative/cellulases (AT1g64390) were induced by this treatment. In the Metabolism group, ethyl acetate sub-fraction treatment activated increased levels of myo-inositol-1-phosphate synthase 2 (AT2g22240) transcripts. Galactinol sythetase genes ATGOLS3 (AT1g09350); ATGOLS2 (AT1g56600) were also up-regulated. Thus, two families of genes that function in the biosynthesis of raffinose oligosaccharide (Myoinositol and Galactinol synthetases) were upregulated by A. nodosum extract. Furthermore, raffinose synthase (AT5g40390) was also up-regulated.

Interestingly, several genes in phenylpropanoid pathway, especially flavonoid synthesis were up-regulated. This included Phenylalanine ammonia lyase 1 (PAL1) and PAL2 (that catalyzes the conversion of phenylalanine to cinnamate); chalcone synthase (CHS), (required for the condensation of 4-coumaroyl-CoA and malonyl-CoA to yield naringenin chalcone); chalcone isomerase (CHI); flavonoid 3′-hydroxylase (F3′H), and dihydroflavonol 4-reductase (DFR). Flavonoids are a diverse group of secondary metabolites with a wide array of biological functions like pigmentation, facilitators of plant-microbe interaction, and reproduction. Flavonoids have also been linked to defense responses against biotic and abiotic stresses, such as pathogens, wounding, and UV light damage. The exact role of flavonoids in salinity stress tolerance is unclear. However, it is possible that flavonoid secondary metabolites will alleviate oxidative stress imposed under high salt concentration.

The transcription factors up-regulated in this category were mainly: zinc finger, myb transcription factors, AP2 domain containing transcription factor. Transcription factors DRE-binding protein (DREB1A)/CRT/DRE-binding factor 3 (CBF3) and DRE-binding protein (DREB1C)/CRT/DRE-binding factor 2 (CBF2) were significantly induced by ethyl acetate sub fraction. The DREB/CBF pathway has been established to be the converging point of NaCl, drought and freezing stress signaling. In the cellular rescue and defense group, glutathione S-transferase (AT5g172200) was shown to be up-regulated.

Category 2: Up-Regulated Genes in Ethyl Actetate Sub Fraction Treatment on Day 5

Category 2 included 257 genes (Table 2) that were up-regulated on day 5 of ethyl acetate sub-fraction treatment. Interestingly, on day 5 of the treatment, the proportion of abiotic stress regulated genes increased to 6.0%. On the other hand, the percentage of genes under transcription factors group decreased on day 5 of the treatment (6.5%) in comparison to day 1 of the treatment. Genes that were up-regulated on day 5 are listed in Table 2. Several group 1 LEA and Group 2 LEA proteins (dehydrins). An ABA induced stress regulation gene, AtHVA22b (AT5g62490), was induced by ethyl acetate sub fraction of A. nodosum. Similarly, Di21 (AT4g15910) and ABA responsive protein-related was also up-regulated. Overall, these genes are involved in abiotic stress and ABA dependent.

TABLE 2
Microarray data for selected genes induced by salinity stress in Arabidopsis thaliana by
ethyl acetate extract treatment after Day 5 of treatment

| | | | |
|---|---|---|---|
| Array | Locus | Fold | |
| Element | Identifier | (log₂) | Annotation |

ABIOTIC STRESS

| | | | |
|---|---|---|---|
| 258347_at | At3g17520 | 15.01491 | late embryogenesis abundant domain-containing protein/LEA |
| | | | domain-containing protein |
| 248352_at | AT5G52300 | 6.511906 | low-temperature-responsive 65 kD protein (LTI65)/desiccation- |
| | | | responsive protein 29B (RD29B) |
| 262128_at | AT1G52690 | 6.839651 | late embryogenesis abundant protein, putative/LEA protein, |
| | | | putative |
| 250648_at | AT5G06760 | 6.689409 | late embryogenesis abundant group 1 domain-containing protein/ |
| | | | LEA group 1 domain-containing protein |
| 266544_at | AT2G35300 | 3.490015 | late embryogenesis abundant group 1 domain-containing protein/ |
| | | | LEA group 1 domain-containing protein |
| 245523_at | AT4G15910 | 4.048004 | drought-responsive protein/drought-induced protein (Di21) |
| 264953_at | AT1G77120 | 2.547603 | alcohol dehydrogenase (ADH) |
| 247437_at | AT5G62490 | 2.470057 | ABA-responsive protein (HVA22b) |
| 258224_at | AT3G15670 | 1.982609 | late embryogenesis abundant protein, putative/LEA protein, |
| | | | putative |
| 263157_at | AT1G54100 | 1.925271 | aldehyde dehydrogenase, putative/antiquitin, putative |
| 256464_at | AT1G32560 | 1.640385 | late embryogenesis abundant group 1 domain-containing protein/ |
| | | | LEA group 1 domain-containing protein |
| 263492_at | AT2G42560 | 1.765456 | late embryogenesis abundant domain-containing protein/LEA |
| | | | domain-containing protein |
| 252137_at | AT3G50980.1 | 4.197867 | dehydrin, putative |
| 247095_at | AT5G66400 | 3.928469 | dehydrin (RAB18) |
| 258498_at | AT3G02480 | 7.061576 | ABA-responsive protein-related |
| 253120_at | AT4G35790 | 1.80686 | phospholipase D delta/PLD delta (PLDDELTA) |

Cellular Organization and Biogenesis

| | | | |
|---|---|---|---|
| 253344_at | AT4G33550 | 4.672816 | protease inhibitor/seed storage/lipid transfer protein (LTP) family |
| | | | protein |
| 266098_at | AT4G15910 | 3.967902 | protease inhibitor/seed storage/lipid transfer protein (LTP) family |
| | | | protein |
| 247718_at | AT5G59310.1 | 3.581127 | lipid transfer protein 4 (LTP4) |
| 265111_at | AT1G62510.1 | 2.871597 | protease inhibitor/seed storage/lipid transfer protein (LTP) family |
| | | | protein |
| 257066_at | AT3G18280 | 1.718598 | protease inhibitor/seed storage/lipid transfer protein (LTP) family |
| | | | protein |
| 247717_at | AT5G59320 | 1.545822 | lipid transfer protein 3 (LTP3) |
| 254189_at | AT4G24000.1 | 3.760111 | cellulose synthase family protein |
| 264514_at | AT1G09500 | 2.724271 | cinnamyl-alcohol dehydrogenase family/CAD family |
| 255787_at | AT2G33590 | 2.152222 | cinnamoyl-CoA reductase family |
| 260568_at | AT2G43570.1 | 2.559195 | chitinase, putative |
| 260561_at | AT2G43580 | 2.334174 | chitinase, putative |
| 247866_at | AT5G57550 | 2.133806 | xyloglucan:xyloglucosyl transferase/xyloglucan |
| | | | endotransglycosylase/endo-xyloglucan transferase (XTR3) |
| 260560_at | AT2G43590 | 1.66276 | chitinase, putative |
| 249767_at | AT5G24090.1 | 2.495187 | acidic endochitinase (CHIB1) |

ENERGY

| | | | |
|---|---|---|---|
| 248236_at | AT5G53870.1 | 3.484386 | plastocyanin-like domain-containing protein |
| 262347_at | AT1G64110 | 2.870212 | AAA-type ATPase family protein |

Metabolism

| | | | |
|---|---|---|---|
| 253373_at | AT4G33150.1 | 4.983652 | lysine-ketoglutarate reductase/saccharopine dehydrogenase |
| | | | bifunctional enzyme |
| 256746_at | AT3G29320 | 1.508099 | glucan phosphorylase, putative |
| 254806_at | AT4G12430 | 1.828979 | trehalose-6-phosphate phosphatase, putative |
| 251137_at | AT5G01300.1 | 3.880385 | phosphatidylethanolamine-binding family protein |
| 250891_at | AT5G04530.1 | 3.798344 | beta-ketoacyl-CoA synthase family protein |
| 266690_at | AT2G19900 | 3.346125 | malate oxidoreductase, putative |
| 252983_at | AT4G37980 | 1.598255 | mannitol dehydrogenase, putative (ELI3-1) |
| 251100_at | AT5G01670 | 1.696478 | aldose reductase, putative |
| 257881_at | AT3G17180 | 2.430443 | serine carboxypeptidase S10 family protein |
| 266278_at | AT2G29300 | 2.380858 | tropinone reductase, putative/tropine dehydrogenase, putative |
| 251191_at | AT3G62590 | 2.129764 | lipase class 3 family protein |
| 253224_at | AT4G34860.1 | 2.051706 | beta-fructofuranosidase, putative/invertase, putative/saccharase, |
| | | | putative/beta-fructosidase, putative |
| 251642_at | AT3G57520 | 2.128627 | alkaline alpha galactosidase, putative |
| 264524_at | AT1G10070 | 1.955038 | branched-chain amino acid aminotransferase 2/branched-chain |
| | | | amino acid transaminase 2 (BCAT2) |
| 265199_s_at | AT2G36780 | 1.738282 | UDP-glucoronosyl/UDP-glucosyl transferase family protein /// |
| | | | UDP-glucoronosyl/UDP-glucosyl transferase family protein |
| 263986_at | AT2G42790 | 1.596132 | citrate synthase, glyoxysomal, putative |
| 251668_at | AT3G57010 | 5.23744 | strictosidine synthase family protein |
| 252068_at | AT3G51440.1 | 2.574864 | strictosidine synthase family protein |
| 250859_at | AT5G04660 | 1.711302 | cytochrome P450, putative |
| 266778_at | AT2G29090 | 1.639061 | cytochrome P450 family protein |
| 259058_at | AT3G03470 | 1.632459 | cytochrome P450, putative |
| 257129_at | AT3G20100 | 1.557462 | cytochrome P450 family protein |
| 255521_at | AT4G02280 | 3.744142 | sucrose synthase, putative/sucrose-UDP glucosyltransferase, |
| | | | putative |
| 262047_at | AT1G80160.1 | 3.467434 | lactoylglutathione lyase family protein/glyoxalase I family protein |
| 259054_at | AT3G03480 | 2.614822 | transferase family protein |
| 258374_at | AT3G14360 | 2.48336 | lipase class 3 family protein |
| 263127_at | AT1G78610 | 2.40754 | Mechano sensitive ion channel domain-containing protein/MS ion |
| | | | channel domain-containing protein |
| 267496_at | AT2G30550 | 1.663507 | lipase class 3 family protein |
| 262122_at | AT1G02790 | 1.63906 | exopolygalacturonase/galacturan 1,4-alpha-galacturonidase |
| | | | (PGA3)/pectinase |

SIGNAL Transduction

| | | | |
|---|---|---|---|
| 252872_at | AT4G40010.1 | 4.47483 | serine/threonine protein kinase, putative |
| 249771_at | AT5G24080.1 | 3.590328 | protein kinase family protein |
| 246922_at | AT5G25110.1 | 2.592862 | CBL-interacting protein kinase 25 (CIPK25) |
| 258652_at | AT3G09910 | 2.277888 | Ras-related GTP-binding protein, putative |
| 264783_at | AT1G08650 | 1.830171 | phosphoenolpyruvate carboxylase kinase |
| 255984_at | AT1G34120 | 1.515543 | inositol polyphosphate 5-phosphatase I (IP5PI) |

TRANSCRIPTION FACTORS

| | | | |
|---|---|---|---|
| 249117_at | AT5G43840.1 | 4.47483 | heat shock transcription factor family protein |
| 260097_at | AT1G73220.1 | 2.520324 | sugar transporter family protein |
| 259618_at | AT1G48000 | 2.411674 | myb family transcription factor |
| 263158_at | AT1G54160.1 | 2.242688 | CCAAT-binding transcription factor (CBF-B/NF-YA) family |
| | | | protein |
| 263128_at | AT1G78600 | 2.14162 | zinc finger (B-box type) family protein |
| 250287_at | AT5G13330 | 2.109079 | AP2 domain-containing transcription factor family protein |
| 264510_at | AT1G09530 | 2.021174 | phytochrome interacting factor 3 (PIF3) |
| 258044_at | AT3G21270 | 2.009734 | Dof-type zinc finger domain-containing protein (ADOF2) |
| 264957_at | AT1G77000 | 1.851241 | F-box family protein |
| 260784_at | AT1G06180 | 1.806539 | myb family transcription factor |
| 255585_at | AT4G01550 | 1.704045 | no apical meristem (NAM) family protein |
| 251084_at | AT5G01520 | 1.557462 | zinc finger (C3HC4-type RING finger) family protein |
| 252408_at | AT3G47600 | 1.541716 | myb family transcription factor (MYB94) |
| 256806_at | AT3G20910 | 1.523143 | CCAAT-binding transcription factor (CBF-B/NF-YA) family |
| | | | protein |
| 267010_at | AT2G39250 | 1.512621 | AP2 domain-containing transcription factor, putative |

CELLULAR RESCUE and DEFENSE

| | | | |
|---|---|---|---|
| 247435_at | AT5G62480.1 | 3.861172 | glutathione S-transferase, putative |
| 263374_at | AT2G20560 | 2.257785 | DNAJ heat shock family protein |
| 261285_at | AT1G35720 | 2.116412 | annexin 1 (ANN1) |
| 258158_at | AT3G17790 | 1.92037 | acid phosphatase type 5 (ACP5) |
| 266294_at | AT2G29500 | 1.583569 | 17.6 kDa class I small heat shock protein (HSP17.6B-CI) |
| 249575_at | AT5G37670 | 1.529129 | 15.7 kDa class I-related small heat shock protein-like (HSP15.7-CI) |
| 249850_at | AT5G23240 | 1.566699 | DNAJ heat shock N-terminal domain-containing protein |
| 265480_at | AT2G15970 | 1.665163 | cold-acclimation protein, putative (FL3-5A3) |

TRANSPORTERS

| | | | |
|---|---|---|---|
| 267080_at | AT2G41190.1 | 2.617936 | amino acid transporter family protein |
| 250506_at | AT5G09930 | 2.152955 | ABC transporter family protein |
| 256757_at | AT3G25620 | 1.538323 | ABC transporter family protein |
| 260163_at | AT1G79900 | 2.061735 | mitochondrial substrate carrier family protein |
| 250161_at | AT5G15240 | 2.047027 | amino acid transporter family protein |
| 245769_at | AT1G30220 | 1.954904 | sugar transporter family protein |
| 264338_at | AT1G70300 | 1.964731 | potassium transporter, putative |
| 256022_at | AT1G58360 | 1.512825 | amino acid permease I (AAP1) |

OTHER GENES

| | | | |
|---|---|---|---|
| 255048_at | AT4G09600 | 4.802326 | gibberellin-regulated protein 3 (GASA3)/gibberellin-responsive |
| | | | protein 3 |
| 245982_at | AT5G13170 | 4.543002 | nodulin MtN3 family protein |
| 256789_at | AT3G13672 | 2.546247 | seven in absentia (SINA) family protein |
| 257271_at | AT3G28007 | 2.408079 | nodulin MtN3 family protein |
| 245703_at | AT5G04380 | 1.898557 | S-adenosyl-L-methionine:carboxyl methyltransferase family protein |
| 248676_at | AT5G48850 | 1.749158 | male sterility MS5 family protein |
| 255575_at | AT4G01430 | 1.73882 | nodulin MtN21 family protein |
| 257893_at | AT3G17000 | 1.732926 | ubiquitin-conjugating enzyme, putative |
| 249979_s_at | AT5G18860 | 1.733286 | inosine-uridine preferring nucleoside hydrolase family protein /// |
| | | | inosine-uridine preferring nucleoside hydrolase family protein |
| 255860_at | AT5G34940 | 1.710485 | glycosyl hydrolase family 79 N-terminal domain-containing protein |
| 264729_at | AT1G22990.1 | 3.242062 | heavy-metal-associated domain-containing protein/copper |
| | | | chaperone (CCH)-related |
| 249917_at | AT5G22460.1 | 3.21146 | esterase/lipase/thioesterase family protein |
| 266462_at | AT2G47770.1 | 3.070209 | benzodiazepine receptor-related |
| 247128_at | AT5G66110 | 2.74753 | heavy-metal-associated domain-containing protein |
| 245076_at | AT2G23170 | 2.689231 | auxin-responsive GH3 family protein |
| 250293_s_at | AT5G13370 | 1.555959 | auxin-responsive GH3 family protein /// auxin-responsive GH3 |
| | | | family protein |
| 261768_at | AT1G15550 | 2.017634 | gibberellin 3-beta-dioxygenase/gibberellin 3 beta-hydroxylase |
| | | | (GA4) |
| 256601_s_at | AT3G28290.1 | 2.251977 | integrin-related protein 14a /// integrin-related protein 14a |
| 267429_at | AT2G34850 | 1.973366 | NAD-dependent epimerase/dehydratase family protein |
| 253172_at | AT4G35060 | 1.936296 | heavy-metal-associated domain-containing protein/copper |
| | | | chaperone (CCH)-related |
| 265796_at | AT2G35730 | 1.705875 | heavy-metal-associated domain-containing protein |
| 262644_at | AT1G62710 | 1.595388 | vacuolar processing enzyme beta/beta-VPE |
| 246861_at | AT5G25890 | 1.513648 | auxin-responsive protein/indoleacetic acid-induced protein 28 |
| | | | (IAA28) |

Unknown genes

| | | | |
|---|---|---|---|
| 247061_at | AT5G66780 | 7.944997 | expressed protein |
| 254823_at | AT4G12580 | 5.4238 | expressed protein |
| 263881_at | AT2G21820.1 | 3.254599 | expressed protein |
| 255527_at | AT4G02360 | 3.217197 | expressed protein |
| 253401_at | AT4G32870 | 2.85058 | expressed protein |
| 249454_at | AT5G39520 | 2.444388 | expressed protein |

We also observed an increase in the expression of phospholipase D delta (AT4g35790). Phospholipase D catalyses the hydrolysis of a structural phospholipid, phosphatidylcholine (PtdCho), and other phospholipids, to form phosphatidic acid (PtdOH) (Liscovitch et al., 2000).

Many of the genes involved in cellular biogenesis, mainly Lipid transfer proteins were induced. (LTPs) are small, abundant basic proteins in higher plants. Ethyl acetate subfraction treatment induced transcription of a number of LTPs (AT4g33550; AT4g15910; AT5g59310.1AT1G62510.1; AT3g18280; AT5g59320). LTPS function by binding fatty acids and by transferring phospholipids between membranes in vitro. Other genes that were induced include GST, Annexin, Glutathione S-transferases, transcription factors like RING zinc finger proteins, MYBs and rd29B.

Category 3: Down-Regulated Genes in Ethyl Actetate Subfraction Treatment on Day 1

Genes that were down-regulated by ethyl acetate fraction on day 1 are listed in Table 3. A number of cellular organization and biogenesis genes were repressed that include cellulose synthase family protein (ATI G55850; AT4G24000); xyloglucan:xyloglucosyl transferase and invertase/pectin methylesterase inhibitor family protein (ATI g62760). A group of genes encoding xyloglucan:xyloglucosyl transferase are also present which are responsible for cell-wall construction in plants. Both these groups of genes are down-regulated by ethyl acetate subfraction treatment Besides, an auxin-responsive gene (AT2g23170) and several heat shock proteins were also repressed.

TABLE 3
Microarray data for selected genes repressed by salinity stress in
Arabidopsis thaliana by ethyl acetate extract treatment after Day 1
of treatment.

| | | | |
|---|---|---|---|
| Array Element | Locus Identifier | Fold (log₂) | Annotation |

CELLULAR ORGANIZATION AND BIOGENESIS

| | | | |
|---|---|---|---|
| 260592_at | AT1G55850 | 0.6467134 | cellulose synthase family protein |
| 254189_at | AT4G24000 | 0.6013953 | cellulose synthase family protein |
| 247866_at | AT5G57550 | 0.4196655 | xyloglucan:xyloglucosyl transferase/ |
| | | | xyloglucan endotransglycosylase/ |
| | | | endo-xyloglucan transferase (XTR3) |
| 262640_at | AT1G62760 | 0.4770471 | invertase/pectin methylesterase |
| | | | inhibitor family protein |

METABOLISM

| | | | |
|---|---|---|---|
| 257216_at | AT3G14990 | 0.658962 | 4-methyl-5(b-hydroxyethyl)-thiazole |
| | | | monophosphate biosynthesis protein, |
| | | | putative |
| 261081_at | AT1G07350 | 0.6519919 | transformer serine/arginine-rich |
| | | | ribonucleoprotein, putative |
| 245076_at | AT2G23170 | 0.6454968 | auxin-responsive GH3 family protein |
| 259418_at | AT1G02390 | 0.6417782 | phospholipid/glycerol acyltransferase |
| | | | family protein |
| 260567_at | AT2G43820 | 0.6411763 | UDP-glucoronosyl/UDP-glucosyl |
| | | | transferase family protein |
| 249869_at | AT5G23050 | 0.6395717 | acyl-activating enzyme 17 (AAE17) |
| 256598_at | AT3G30180 | 0.6338174 | cytochrome P450, putative |
| 258063_at | AT3G14620 | 0.6321929 | cytochrome P450, putative |
| 247348_at | AT5G63810 | 0.6255499 | beta-galactosidase, putative/lactase, |
| | | | putative |
| 258336_at | AT3G16050 | 0.6242512 | stress-responsive protein, putative |
| 253101_at | AT4G37430, | 0.6178195 | cytochrome P450 81F1 (CYP81F1) |
| | | | (CYP91A2) |
| 259694_at | AT1G63180 | 0.5897521 | UDP-glucose 4-epimerase, putative/ |
| | | | UDP-galactose 4-epimerase, putative/ |
| | | | Galactowaldenase, putative |
| 248918_at | AT5G45890 | 0.587878 | senescence-specific SAG12 protein |
| | | | (SAG12)/cysteine proteinase, |
| | | | putative |
| 266578_at | AT2G23910 | 0.5510421 | cinnamoyl-CoA reductase-related |
| 254764_at | AT4G13250 | 0.5501392 | short-chain dehydrogenase/reductase |
| | | | (SDR) family protein |
| 255943_at | AT1G22370 | 0.5122407 | UDP-glucoronosyl/UDP-glucosyl |
| | | | transferase family protein |

SIGNAL TRANSDUCTION

| | | | |
|---|---|---|---|
| 266743_at | AT2G02990 | 0.6475001 | ribonuclease 1 (RNS1) |
| 248429_at | AT5G51770 | 0.6437611 | protein kinase family protein |
| 251432_at | AT3G59820 | 0.6388801 | calcium-binding mitochondrial |
| | | | protein-related |
| 256965_at | AT3G13450 | 0.6259579 | 2-oxoisovalerate dehydrogenase/ |
| | | | 3-methyl-2-oxobutanoate |
| | | | dehydrogenase/branched-chain |
| | | | alpha-keto acid dehydrogenase E1 |
| | | | beta subunit (DIN4) |
| 248046_at | AT5G56040 | 0.5736343 | leucine-rich repeat protein kinase, |
| | | | putative |
| 246028_at | AT5G21170 | 0.5517346 | 5′-AMP-activated protein kinase |
| | | | beta-2 subunit, putative |

TRANSCRIPTION FACTORS

| | | | |
|---|---|---|---|
| 251575_at | AT3G58120 | 0.6544735 | bZIP transcription factor family |
| | | | protein |
| 260431_at | AT1G68190 | 0.633786 | zinc finger (B-box type) family |
| | | | protein |
| 249234_at | AT5G42200 | 0.6324005 | zinc finger (C3HC4-type RING |
| | | | finger) family protein |
| 249862_at | AT5G22920 | 0.631574 | zinc finger (C3HC4-type RING |
| | | | finger) family protein |
| 252475_s_at | AT5G59570 | 0.6210956 | myb family transcription factor /// |
| | | | myb family transcription factor |
| 258133_at | AT3G24500 | 0.6148897 | ATMBF1C/MBF1C |
| | | | (MULTIPROTEIN BRIDGING |
| | | | FACTOR 1C); DNA binding/ |
| | | | transcription coactivator/ |
| | | | transcription factor |
| 266839_at | AT2G25930 | 0.6140176 | ELF3 (EARLY FLOWERING 3) |
| 248606_at | AT5G49450 | 0.5904502 | bZIP family transcription factor |
| 260266_at | AT1G68520 | 0.5843768 | zinc finger (B-box type) family |
| | | | protein |
| 261265_at | AT1G26800 | 0.5620626 | zinc finger (C3HC4-type RING |
| | | | finger) family protein |
| 252367_at | AT3G48360 | 0.3535679 | BT2 (BTB and TAZ domain |
| | | | protein 2); protein binding/ |
| | | | transcription regulator |
| 259244_at | AT3G07650 | 0.3175566 | COL9 (CONSTANS-LIKE 9); |
| | | | transcription factor/zinc ion |
| | | | binding |
| 257985_at | AT3G20810 | 0.2453973 | transcription factor jumonji (jmjC) |
| | | | domain-containing protein |

CELLULAR RESCUE AND DEFENSE

| | | | |
|---|---|---|---|
| 263374_at | AT2G20560 | 0.6543058 | DNAJ heat shock family protein |
| 265471_at | AT2G37130 | 0.6189507 | peroxidase 21 (PER21) (P21) |
| | | | (PRXR5) |
| 249850_at | AT5G23240 | 0.6070001 | DNAJ heat shock N-terminal |
| | | | domain-containing protein |
| 256245_at | AT3G12580 | 0.5724005 | heat shock protein 70, putative/ |
| | | | HSP70, putative |
| 248332_at | AT5G52640 | 0.5616923 | heat shock protein 81-1 (HSP81-1)/ |
| | | | heat shock protein 83 (HSP83) |
| 258957_at | AT3G01420 | 0.4513215 | pathogen-responsive alpha- |
| | | | dioxygenase, putative |
| 248045_at | AT5G56030 | 0.6028267 | heat shock protein 81-2 (HSP81-2) |

UNKNOWN GENES

| | | | |
|---|---|---|---|
| 253630_at | AT4G30490 | 0.6661879 | AFG1-like ATPase family protein |
| 254574_at | AT4G19430 | 0.6644498 | expressed protein |
| 267178_at | AT2G37750 | 0.6620409 | expressed protein |
| 261608_at | AT1G49650 | 0.6582341 | cell death associated protein- |
| | | | related |
| 263210_at | AT1G10585 | 0.6547906 | expressed protein |
| 260933_at | AT1G02470 | 0.6540852 | expressed protein |
| 265478_at | AT2G15890 | 0.6520669 | expressed protein |
| 258647_at | AT3G07870 | 0.649577 | F-box family protein |
| 245433_at | AT4G17110 | 0.6486495 | expressed protein |
| 247443_at | AT5G62720 | 0.6427957 | integral membrane HPP family |
| | | | protein |
| 265387_at | AT2G20670 | 0.6365452 | expressed protein |
| 260688_at | AT1G17665 | 0.629737 | expressed protein |
| 262229_at | AT1G68620 | 0.6251304 | expressed protein |
| 249454_at | AT5G39520 | 0.6247977 | expressed protein |
| 245434_at | AT4G17120 | 0.621736 | expressed protein |
| 253322_at | AT4G33980 | 0.6192145 | expressed protein |
| 258262_at | AT3G15770 | 0.6189757 | expressed protein |
| 247293_at | AT5G64510 | 0.6097934 | expressed protein |
| 249190_at | AT5G42750 | 0.588055 | expressed protein |
| 249377_at | AT5G40690 | 0.5853797 | expressed protein |
| 253874_at | AT4G27450 | 0.5333842 | expressed protein |
| 249174_at | AT5G42900 | 0.5323932 | expressed protein |
| 258939_at | AT3G10020 | 0.5271035 | expressed protein |
| 258225_at | AT3G15630 | 0.5155414 | expressed protein |
| 249923_at | AT5G19120 | 0.4857222 | expressed protein |
| 267364_at | AT2G40080 | 0.4846057 | expressed protein |
| 251293_at | AT3G61930 | 0.4634457 | expressed protein |
| 257615_at | AT3G26510 | 0.5881068 | octicosapeptide/Phox/Bem1p |
| | | | (PB1) domain-containing protein |
| 259382_s_at | AT3G16430 | 0.5787531 | jacalin lectin family protein /// |
| | | | jacalin lectin family protein |
| 259502_at | AT1G15670 | 0.5692633 | kelch repeat-containing F-box |
| | | | family protein |
| 260101_at | AT1G73260 | 0.5661098 | trypsin and protease inhibitor |
| | | | family protein/Kunitz family |
| | | | protein |
| 256060_at | AT1G07050 | 0.5603096 | CONSTANS-like protein-related |
| 253024_at | AT4G38080 | 0.5514374 | hydroxyproline-rich glycoprotein |
| | | | family protein |
| 267238_at | AT2G44130 | 0.5333408 | kelch repeat-containing F-box |
| | | | family protein |
| 249174_at | AT5G42900 | 0.5323932 | expressed protein |
| 245668_at | AT1G28330 | 0.377387 | dormancy-associated protein, |
| | | | putative (DRM1) |

OTHER GENES

| | | | |
|---|---|---|---|
| 259037_at | AT3G09350 | 0.6384012 | armadillo/beta-catenin repeat |
| | | | family protein |
| 250217_at | AT5G14120 | 0.633485 | nodulin family protein |
| 245136_at | AT2G45210 | 0.6271569 | auxin-responsive protein-related |
| 255345_at | AT4G04460 | 0.6041092 | aspartyl protease family protein |
| 260427_at | AT1G72430 | 0.5536881 | auxin-responsive protein-related |
| 252698_at | AT3G43670 | 0.5290776 | copper amine oxidase, putative |
| 267461_at | AT2G33830 | 0.5287762 | dormancy/auxin associated family |
| | | | protein |
| 246114_at | AT5G20250 | 0.5174247 | raffinose synthase family protein/ |
| | | | seed imbibition protein, putative |
| | | | (din10) |
| 247668_at | AT5G60100 | 0.4680666 | pseudo-response regulator 3 |
| | | | (APRR3) |
| 245076_at | AT2G23170 | 0.6454968 | auxin-responsive GH3 family |
| | | | protein |

### Category 4: Down-Regulated Genes in Ethyl Actetate Sub Fraction Treatment on Day 5

The classification of repressed genes on day 5 of ethyl acetate fraction treatment. Notably, only a small subset of abiotic factors (1.6%) was down-regulated. Table 4 lists the genes that were down-regulated by ethyl acetate fraction treatment on day 5. Many of the abiotic stress genes, including AT4g19120; AT3g30775 were down-regulated. It has been shown that reciprocal regulation of P5CS and PDH genes appears to regulate the concentration of proline under osmotic stress. The induction of PDH by proline, however, was inhibited by salt stress (Peng, Z et al., 1996). In the cellular biogenesis group, cellulose synthetase and a few pectinesterases were inhibited. Similarly, transcripts of a wall-associated kinase (WAK1), RNA binding proteins AT5G61030 and AT4G39260 were also reduced.

TABLE 4
Microarray data for selected genes repressed by salinity stress in
Arabidopsis thaliana by ethyl acetate extract treatment after Day 5
of treatment

| | | | |
|---|---|---|---|
| Array | Locus | Fold | |
| Element | Identifier | (log₂) | Annotation |

ABIOTIC STRESS

| | | | |
|---|---|---|---|
| 254563_at | AT4G19120 | 0.647701 | early-responsive to dehydration stress protein (ERD3) |
| 257315_at | AT3G30775 | 0.5572566 | proline oxidase, mitochondrial/osmotic stress-responsive proline |
| | | | dehydrogenase (POX) (PRO1) (ERD5) |
| 245736_at | AT1G73330 | 0.5882807 | protease inhibitor, putative (DR4) |

Cellular Organization and Biogenesis

| | | | |
|---|---|---|---|
| 254185_at | AT4G23990 | 0.6028183 | cellulose synthase family protein |
| 258750_at | AT3G05910 | 0.64114 | pectinacetylesterase, putative |
| 261055_at | AT1G01300 | 0.6151036 | aspartyl protease family protein |
| 248419_at | AT5G51550 | 0.5840732 | phosphate-responsive 1 family protein |
| 258764_at | AT3G10720 | 0.5772374 | pectinesterase, putative |
| 262225_at | AT1G53840 | 0.6164894 | pectinesterase family protein |
| | | | ENERGY |
| 259507_at | AT1G43910 | 0.5697036 | AAA-type ATPase family protein |

Metabolism

| | | | |
|---|---|---|---|
| 266338_at | AT2G32400 | 0.6572218 | glutamate receptor family protein (GLR3.7) (GLR5) |
| 259763_at | AT1G77630 | 0.6472076 | peptidoglycan-binding LysM domain-containing protein |
| 262414_at | AT1G49430 | 0.6407058 | long-chain-fatty-acid--CoA ligase/long-chain acyl-CoA synthetase |
| 264339_at | AT1G70290 | 0.6406543 | trehalose-6-phosphate synthase, putative |
| 248404_at | AT5G51460 | 0.5753056 | trehalose-6-phosphate phosphatase (TPPA |
| 264956_at | AT1G76990 | 0.5620685 | ACT domain containing protein |
| 254687_at | AT4G13770 | 0.6489285 | cytochrome P450 family protein |
| 262793_at | AT1G13110 | 0.6155948 | cytochrome P450 71B7 (CYP71B7) |
| 246949_at | AT5G25140 | 0.6032137 | cytochrome P450 family protein |
| 266996_at | AT2G34490 | 0.5750135 | cytochrome P450 family protein |
| 245676_at | AT1G56670 | 0.6613705 | GDSL-motif lipase/hydrolase family protein |
| 267162_s_at | AT2G37690 | 0.6585301 | phosphoribosylaminoimidazole carboxylase family protein/AIR |
| | | | carboxylase family protein /// phosphoribosylaminoimidazole |
| | | | carboxylase family protein/AIR carboxylase family protein |
| 267126_s_at | AT2G23600 | 0.6117982 | hydrolase, alpha/beta fold family protein /// hydrolase, alpha/beta |
| | | | fold family protein |
| 254835_s_at | AT4G12320 | 0.6572362 | cytochrome P450, putative /// cytochrome P450, putative |
| 254163_s_at | AT4G24340 | 0.6179798 | phosphorylase family protein /// phosphorylase family protein |
| 261804_at | AT1G30530 | 0.6109135 | UDP-glucoronosyl/UDP-glucosyl transferase family protein |
| 264100_at | AT1G78970 | 0.608777 | lupeol synthase (LUP1)/2,3-oxidosqualene-triterpenoid cyclase |
| 254328_at | AT4G22570 | 0.5959715 | adenine phosphoribosyltransferase, putative |
| 249777_at | AT5G24210 | 0.5699643 | lipase class 3 family protein |
| 267138_s_at | AT2G38230.1 | 0.6213904 | ethylene-responsive protein, putative /// ethylene-responsive protein, |
| | | | putative |
| 255692_at | AT4G00400 | 0.5554115 | phospholipid/glycerol acyltransferase family protein |
| 246330_at | AT3G43600 | 0.6589094 | aldehyde oxidase, putative |

SIGNAL Transduction

| | | | |
|---|---|---|---|
| 261479_at | AT1G14380 | 0.6509323 | calmodulin-binding family protein |
| 264693_at | AT1G69970 | 0.6496384 | CLE26, putative |
| 256769_at | AT3G13690 | 0.6486765 | protein kinase family protein |
| 259560_at | AT1G21270 | 0.6477601 | wall-associated kinase 2 (WAK2) |
| 260774_at | AT1G78290 | 0.6378505 | serine/threonine protein kinase, putative |
| 259561_at | AT1G21250 | 0.6225038 | wall-associated kinase 1 (WAK1) |
| 252280_at | AT3G49260 | 0.6222309 | calmodulin-binding family protein |
| 245765_at | AT1G33600 | 0.6144454 | leucine-rich repeat family protein |
| 265467_at | AT2G37050 | 0.6126747 | leucine-rich repeat family protein/protein kinase family protein |
| 259348_at | AT3G03770 | 0.6075288 | leucine-rich repeat transmembrane protein kinase, putative |
| 247383_at | AT5G63410 | 0.6046734 | leucine-rich repeat transmembrane protein kinase, putative |
| 266078_at | AT2G40670 | 0.6002468 | two-component responsive regulator/response regulator 16 |
| | | | (ARR16) |
| 259080_at | AT3G04910 | 0.5973995 | protein kinase family protein |
| 251714_at | AT3G56370 | 0.5956617 | leucine-rich repeat transmembrane protein kinase, putative |
| 255344_s_at | AT4G04570 | 0.6244325 | protein kinase family protein /// protein kinase family protein |
| 253949_at | AT4G26780 | 0.594573 | co-chaperone grpE family protein |
| 256516_at | AT1G66150 | 0.5707533 | leucine-rich repeat protein kinase, putative (TMK1) |
| 247153_at | AT5G65700 | 0.5580592 | leucine-rich repeat transmembrane protein kinase, putative |
| 257964_at | AT3G19850 | 0.5827569 | phototropic-responsive NPH3 family protein |
| 257206_at | AT3G16530 | 0.5523409 | legume lectin family protein |

TRANSCRIPTION FACTORS

| | | | |
|---|---|---|---|
| 251365_at | AT3G61310 | 0.6660593 | DNA-binding family protein |
| 252033_at | AT3G51950 | 0.6645191 | zinc finger (CCCH-type) family protein/RNA recognition motif |
| | | | (RRM)-containing protein |
| 251374_at | AT3G60390 | 0.6628526 | homeobox-leucine zipper protein 3 (HAT3)/HD-ZIP protein 3 |
| 255636_at | AT4G00730 | 0.662729 | anthocyaninless2 (ANL2) |
| 247575_at | AT5G61030 | 0.6626609 | RNA-binding protein, putative |
| 261254_at | AT1G05805 | 0.6612244 | basic helix-loop-helix (bHLH) family protein |
| 253996_at | AT4G26110 | 0.6570448 | nucleosome assembly protein (NAP), putative |
| 265718_at | AT2G03340 | 0.6538019 | WRKY family transcription factor |
| | | 0.6469632 | |
| 260664_at | AT1G19510 | 0.6414992 | myb family transcription factor |
| 252885_at | AT4G39260 | 0.6268705 | glycine-rich RNA-binding protein 8 (GRP8) (CCR1) |
| 260395_at | AT1G69780 | 0.6146252 | homeobox-leucine zipper protein 13 (HB-13)/HD-ZIP transcription |
| | | | factor 13 |
| 253806_at | AT4G28270 | 0.6070816 | zinc finger (C3HC4-type RING finger) family protein |
| 261603_at | AT1G49600 | 0.6065655 | RNA-binding protein 47 (RBP47), putative |
| 250582_at | AT5G07580 | 0.6017309 | ethylene-responsive element-binding family protein |
| 250167_at | AT5G15310 | 0.6046402 | myb family transcription factor |
| 245183_at | AT5G12440 | 0.5924099 | zinc finger (CCCH-type) family protein |
| 245925_at | AT5G28770 | 0.5880778 | bZIP transcription factor family protein |
| 253061_at | AT4G37610 | 0.5638107 | TAZ zinc finger family protein/BTB/POZ domain-containing |
| | | | protein |
| 246522_at | AT5G15830 | 0.5627193 | bZIP transcription factor family protein |
| 260618_at | AT1G53230 | 0.5603034 | TCP family transcription factor 3 (TCP3) |
| 258890_at | AT3G05690 | 0.547895 | CCAAT-binding transcription factor (CBF-B/NF-YA) family protein |
| 252483_at | AT3G46600 | 0.633563 | scarecrow transcription factor family protein |
| 247029_at | AT5G67190 | 0.6326514 | AP2 domain-containing transcription factor, putative |
| 245029_at | AT2G26580 | 0.6236421 | plant-specific transcription factor YABBY family protein |
| 251373_at | AT3G60530 | 0.6207959 | zinc finger (GATA type) family protein |
| 250694_at | AT5G06710 | 0.612039 | homeobox-leucine zipper protein 14 (HAT14)/HD-ZIP protein 14 |
| 260070_at | AT1G73830 | 0.6469632 | basic helix-loop-helix (bHLH) family protein |

CELLULAR RESCUE and DEFENSE

| | | | |
|---|---|---|---|
| 252712_at | AT3G43800 | 0.5759795 | glutathione S-transferase, putative |
| 259102_at | AT3G11660 | 0.5984216 | harpin-induced family protein/HIN1 family protein/harpin- |
| | | | responsive family protein |

TRANSPORTERS

| | | | |
|---|---|---|---|
| 250248_at | AT5G13740 | 0.6640223 | sugar transporter family protein |
| 261143_at | AT1G19770 | 0.6625168 | purine permease-related |
| 245399_at | AT4G17340 | 0.6549112 | DELTA-TIP2/TIP2; 2 (tonoplast intrinsic protein 2; 2); water channel/ |
| | | | major intrinsic family protein/MIP family protein |
| 247923_at | AT5G57490 | 0.6536791 | porin, putative |
| 262797_at | AT1G20840 | 0.6239806 | transporter-related |
| 259185_at | AT3G01550 | 0.6186855 | triose phosphate/phosphate translocator, putative |
| 245891_at | AT5G09220 | 0.5838115 | amino acid permease 2 (AAP2) |
| 251906_at | AT3G53720 | 0.5760856 | cation/hydrogen exchanger, putative (CHX20) |
| 267305_at | AT2G30070 | 0.5658897 | potassium transporter (KUP1) |
| 264348_at | AT1G12110 | 0.564492 | nitrate/chlorate transporter (NRT1.1) (CHL1) |
| 262883_at | AT1G64780 | 0.5623503 | ammonium transporter 1, member 2 (AMT1.2) |
| 262456_at | AT1G11260 | 0.5612672 | glucose transporter (STP1) |
| 263319_at | AT2G47160 | 0.5597867 | anion exchange family protein |
| 247304_at | AT5G63850 | 0.5419114 | amino acid transporter 4, putative (AAP4) |
| 252594_at | AT3G45680 | 0.6578252 | proton-dependent oligopeptide transport (POT) family protein |
| 250123_at | AT5G16530 | 0.6332869 | auxin efflux carrier family protein |
| 254862_at | AT4G12030 | 0.6125951 | bile acid:sodium symporter family protein |
| 259680_at | AT1G77690 | 0.604502 | amino acid permease, putative |
| 263867_at | AT2G36830 | 0.5610635 | major intrinsic family protein/MIP family protein |

Unknown genes

| | | | |
|---|---|---|---|
| 253165_at | AT4G35320 | 0.64601 | expressed protein |
| 253891_at | AT4G27720 | 0.5921742 | expressed protein |
| 261247_at | AT1G20070 | 0.5855067 | expressed protein |
| 249190_at | AT5G42750 | 0.540648 | expressed protein |
| 256675_at | AT3G52170 | 0.6653361 | expressed protein |
| 257076_at | AT3G19680 | 0.6647934 | expressed protein |
| 256396_at | AT3G06150 | 0.6637115 | expressed protein |
| 263632_at | AT2G04795 | 0.6620487 | expressed protein |
| 257860_at | AT3G13062 | 0.6602042 | expressed protein |
| 245501_at | AT4G15620 | 0.6581931 | integral membrane family protein |
| 245229_at | AT4G25620 | 0.6570833 | hydroxyproline-rich glycoprotein family protein |
| 265726_at | AT2G32010 | 0.6551995 | endonuclease/exonuclease/phosphatase family protein |
| 251793_at | AT3G55580 | 0.6540455 | regulator of chromosome condensation (RCC1) family protein |
| 259520_at | AT1G12320 | 0.6510951 | expressed protein |
| 248238_at | AT5G53900 | 0.6482072 | expressed protein |
| 265716_at | AT2G03350 | 0.6473129 | expressed protein |
| 261377_at | AT1G18850 | 0.6417601 | expressed protein |
| 267199_at | AT2G30990 | 0.6411912 | expressed protein |
| 247417_at | AT5G63040 | 0.6409489 | expressed protein |
| 250107_at | AT5G15330 | 0.6354472 | SPX (SYG1/Pho81/XPR1) domain-containing protein |
| 261439_at | AT1G28395 | 0.632307 | expressed protein |
| 248302_at | AT5G53160 | 0.6304484 | expressed protein |
| 250696_at | AT5G06790 | 0.6289911 | expressed protein |
| 266473_at | AT2G31110 | 0.6287217 | expressed protein |
| 250307_at | AT5G12170 | 0.628706 | expressed protein |
| 258460_at | AT3G17330 | 0.6281968 | expressed protein |
| 264609_at | AT1G04530 | 0.6182555 | expressed protein |
| 264590_at | AT2G17710 | 0.6173761 | expressed protein |
| 262868_at | AT1G64980 | 0.6157789 | expressed protein |
| 262446_at | AT1G49310 | 0.6151057 | expressed protein |
| 245479_at | AT4G16140 | 0.6147034 | proline-rich family protein |
| 255224_at | AT4G05400 | 0.608637 | expressed protein |
| 252471_at | AT3G46820 | 0.6040995 | serine/threonine protein phosphatase PP1 isozyme 5 (TOPP5)/ |
| | | | phosphoprotein phosphatase 1 |
| 256570_at | AT3G19540 | 0.6035768 | expressed protein |
| 262094_at | AT1G56110 | 0.5976843 | nucleolar protein Nop56, putative |
| 263662_at | AT1G04430 | 0.5972668 | dehydration-responsive protein-related |
| 264160_at | AT1G65450 | 0.5951164 | transferase family protein |
| 245984_at | AT5G13090 | 0.5952409 | expressed protein |
| 265025_at | AT1G24575 | 0.5896628 | expressed protein |
| 249378_at | AT5G40450 | 0.5886615 | expressed protein |
| 248186_at | AT5G53880 | 0.588162 | expressed protein |
| 261056_at | AT1G01360 | 0.5767976 | expressed protein |
| 251355_at | AT3G61100 | 0.5742316 | expressed protein |
| 266474_at | AT2G31110 | 0.5736725 | expressed protein |
| 251476_at | AT3G59670 | 0.5734383 | expressed protein |
| 267339_at | AT2G39870 | 0.5630156 | expressed protein |

OTHER GENES

| | | | |
|---|---|---|---|
| 244937_at | ATCG01110 | 0.658965 | 49 KDa plastid NAD(P)H dehydrogenase subunit H protein |
| 267288_at | AT2G23680 | 0.6535307 | stress-responsive protein, putative |
| 266460_at | AT2G47930 | 0.6526838 | hydroxyproline-rich glycoprotein family protein |
| 264605_at | AT1G04550 | 0.591109 | auxin-responsive protein/indoleacetic acid-induced protein 12 |
| | | | (IAA12) |
| 265872_at | AT2G01670 | 0.6485412 | MutT/nudix family protein |
| 250546_at | AT5G08180 | 0.6573014 | ribosomal protein L7Ae/L30e/S12e/Gadd45 family protein |
| 256862_at | AT3G23940 | 0.6160713 | dehydratase family |
| 258549_at | AT3G06930 | 0.6563085 | protein arginine N-methyltransferase family protein |
| 264770_at | AT1G23030 | 0.6530662 | armadillo/beta-catenin repeat family protein/U-box domain- |
| | | | containing protein |
| 251740_at | AT3G56070 | 0.6414133 | peptidyl-prolyl cis-trans isomerase, putative/cyclophilin, putative/ |
| | | | rotamase, putative |
| 262162_at | AT1G78020 | 0.5921358 | senescence-associated protein-related |
| 250633_at | AT5G07460 | 0.5904073 | peptide methionine sulfoxide reductase, putative |
| 259955_s_at | AT1G75080 | 0.5876056 | brassinosteroid signalling positive regulator, putative /// |
| | | | brassinosteroid signalling positive regulator, putative |
| 267517_at | AT2G30520 | 0.5829322 | signal transducer of phototropic response (RPT2) |
| 260101_at | AT1G73260 | 0.5764861 | trypsin and protease inhibitor family protein/Kunitz family protein |
| 257748_at | AT3G18710 | 0.6546239 | U-box domain-containing protein |
| 257300_at | AT3G28080 | 0.5693235 | nodulin MtN21 family protein |
| 265962_at | AT2G37460 | 0.5684019 | nodulin MtN21 family protein |
| 263679_at | AT1G59990 | 0.5578415 | DEAD/DEAH box helicase, putative (RH22) |
| 260739_at | AT1G15000 | 0.5560959 | serine carboxypeptidase S10 family protein |
| 248467_at | AT5G50800 | 0.5472308 | nodulin MtN3 family protein |
| 252992_at | AT4G38520 | 0.6484653 | protein phosphatase 2C family protein/PP2C family protein |
| 248427_at | AT5G51750 | 0.6480575 | subtilase family protein |
| 255578_at | AT4G01450 | 0.6418294 | nodulin MtN21 family protein |
| 251853_at | AT3G54790 | 0.6406978 | armadillo/beta-catenin repeat family protein/U-box domain- |
| | | | containing protein |
| 253566_at | AT4G31210 | 0.6394941 | DNA topoisomerase family protein |
| 255127_at | AT4G08300 | 0.6350847 | nodulin MtN21 family protein |
| 245063_at | AT2G39795 | 0.6219979 | mitochondrial glycoprotein family protein/MAM33 family protein |
| 264340_at | AT1G70280 | 0.6108837 | NHL repeat-containing protein |
| 252327_at | AT3G48740 | 0.6102671 | nodulin MtN3 family protein |
| 258155_at | AT3G18130 | 0.6089905 | guanine nucleotide-binding family protein/activated protein kinase |
| | | | C receptor (RACK1) |
| 258708_at | AT3G09580 | 0.6023964 | amine oxidase family protein |
| 257288_at | AT3G29670 | 0.5823003 | transferase family protein |
| 259444_at | AT1G02370 | 0.5748528 | pentatricopeptide (PPR) repeat-containing protein |
| 256222_at | AT1G56210 | 0.5745925 | copper chaperone (CCH)-related |
| 265672_at | AT2G31980 | 0.6177624 | cysteine proteinase inhibitor-related |
| 246701_at | AT5G28020 | 0.6582037 | cysteine synthase, putative/O-acetylserine (thiol)-lyase, putative/ |
| | | | O-acetylserine sulfhydrylase, putative |
| 261406_at | AT1G18800 | 0.5675847 | nucleosome assembly protein (NAP) family protein |

Validation of Microarrays Using Semi-Quantitative RT-PCR:

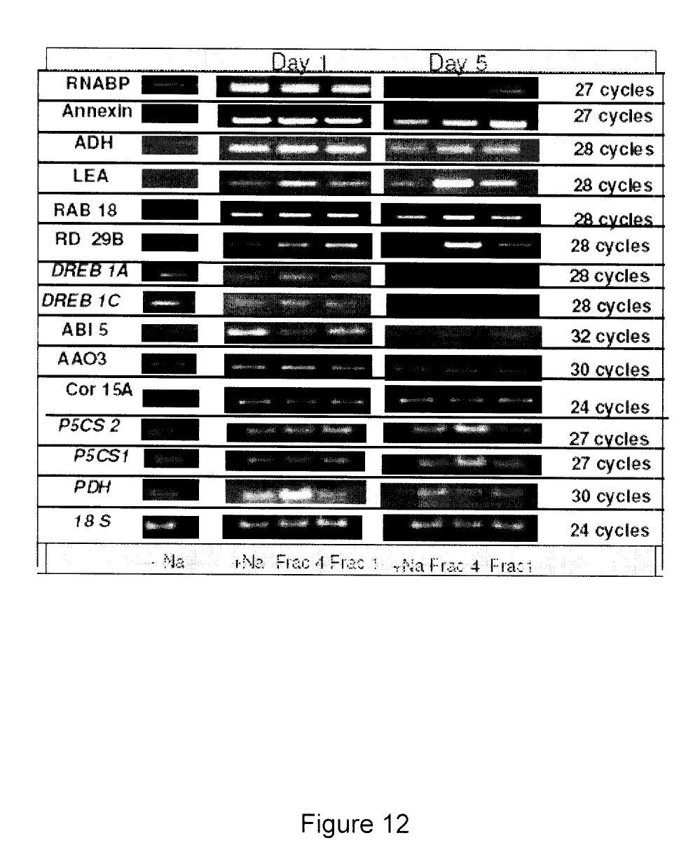

To confirm the validity of the microarray data, we conducted semi quantitative RT-PCR of selected genes that showed differential expression upon treatment with ethyl actetate subfraction. 18S rDNA were used as a control to ensure loading of equal concentrations of cDNA. The first set of genes that we studied were those involved in proline metabolism. We did not find increase in expression of P5CS1 and P5CS2 on day 1 of NaCl treatment. On the other hand, in day 5, transcripts of P5CS1 and P5CS2 increased. As seen in the microarray results, RT-PCR also confirmed that PDH expression levels decreased upon treatment with ethyl acetate extract fractions (FIG. 12).

Salt stress tolerance is promoted by the upregulation of stress responsive genes. We selected several stress genes as shown by Microarray result and other stress induced genes (DREB 2A, DREB 1A, 1C, Cor 15A, RD 29A, RD 29B, RAB and LEA) and transcript levels were analyzed using RT-PCR. DREB 2A, encoding a transcription factor activated in the early stages of abiotic stress, was significantly induced on day 1 than on day 5 of the NaCl treatment. However, there were no clear differences between treatment with ethyl acetate extract fractions and NaCl treated plants. A similar expression profile was observed for COR15A, which encodes a chloroplast targeted LEA-protein (Artus et al., 1996). DREB 1A, 1C increased in day 1 of the extract treatment in comparison to NaCl treated controls like in microarray. rD29A and rd29B (Responsive to desiccation), which are tandemly organized in the Arabidopsis genome have been shown to differ in the expression kinetics during salt stress treatments. While rd29A mRNA levels could be detected in the early stages of salt stress, rd29B mRNA accumulated at a much slower rate. We also obtained similar results after NaCl treatment. rd29A mRNA accumulation was much more pronounced than rd29B at day 1 of NaCl treatment. We found increased expression of rd29A and rd29B mRNA in treatments with ethyl acetate extract fractions than in NaCl treated plants. rd 29B showed increased expression on day 5 of the treatment and the pattern mirrored the microarray results. Again, RAB 18 and LEA, Di 21, RNABP, Annexin and AmmT, which showed increased expression in microarrays, were also confirmed by RT-PCR.

Effect of Extract on the Alleviation of Salt Stress in a Variety of Crop Plants:

Seeds of pea, lettuce, mung bean, cucumber, cauliflower, barley, and cabbages were placed in Petri dishes on filter paper soaked with water (control), 150 mM NaCl or 150 Mm NaCl+organic fraction of A. nodosum. For each treatment there were 5 replications. The Petri dishes were incubated and percent germination was observed periodically (Table 5).

TABLE 5
Organic components of A. nodosum extract alleviates salt stress
(by increased germination and vigour) in a number of crop plants

| | | NaCl | NaCl (200 mM) + A. nodosum |
|---|---|---|---|
| Crop | Control | (200 mM) | extract |
| Cauliflower | 96 | 76 | 92 |
| Pea | 96 | 56 | 74 |
| Lettuce | 96 | 22 | 42 |
| Barley | 62 | 20 | 30 |
| Mungbean | 62 | 20 | 30 |

Induction of Salt Tolerance by A. nodosum in Lettuce and Beet

Abiotic stresses exert oxidative damage through reactive oxygen species (ROS) causing harm to cellular components including membrane lipids (Smirnoff, 1995). High salinity has shown to be the most severe factor, limiting plant growth in the salt affected areas. Saline conditions have shown to increase ROS causing membrane damage and hence it has been the major cause of the cellular toxicity by salinity in C3 and C4 plants (Greenway and Munns, 1980; Hasegawa et al., 2000). Antioxidant enzymes are related to the resistance of various abiotic stresses including salinity. Estimating antioxidant enzyme response of glycophytes, garden lettuce (Lactuca sativa L.) and sugarbeet (Beta vulgaris L.) to salt treatments can provide important clues to the mechanisms of saline stress tolerance in crop plants. Among several antioxidant enzymes, catalase has been found to be greatly enhanced under saline stress in barley (Kim et al., 2005).

Extracts from Ascophyllum nodosum were tested to ascertain whether they could enhance plant antioxidant enzyme response to salt stress and retain chlorophyll levels in saline stress.

Estimation of Catalase activity: Circular leaf discs of (diameter 2-3 cm) sugar beet and lettuce weighing ˜200 mg were floated in Petridishes containing 20 ml of 1.0 g/L 1186 MeOH extracted Soluble Seaweed Extract Powder (SSEP), 1186 CHCl₃ extracted SSEP, 1186 Ethyl acetate extracted SSEP, NaCl 100 and 150 mM and in combinations. The treatments were replicated 4 times and the experimental design followed was completely randomized design. The treatments include:

1. Control 1(NaCl 100 mM)

2. Control 2 (NaCl 150 mM)

3. 1186 Me-OH extracted SSEP

4. 1186 CHCl₃ extracted SSEP

5. 1186 Ethyl acetate extracted SSEP

6. 100 mM NaCl+1186 Me-OH extracted SSEP

7. 100 mM NaCl+1186 CHCl₃ extracted SSEP

8. 100 mM NaCl+1186 Ethyl acetate extracted SSEP

9. 150 mM NaCl+1186 Me-OH extracted SSEP

10. 150 mM NaCl+1186 CHCl₃ extracted SSEP

11. 150 mM NaCl+1186 Ethyl acetate extracted SSEP

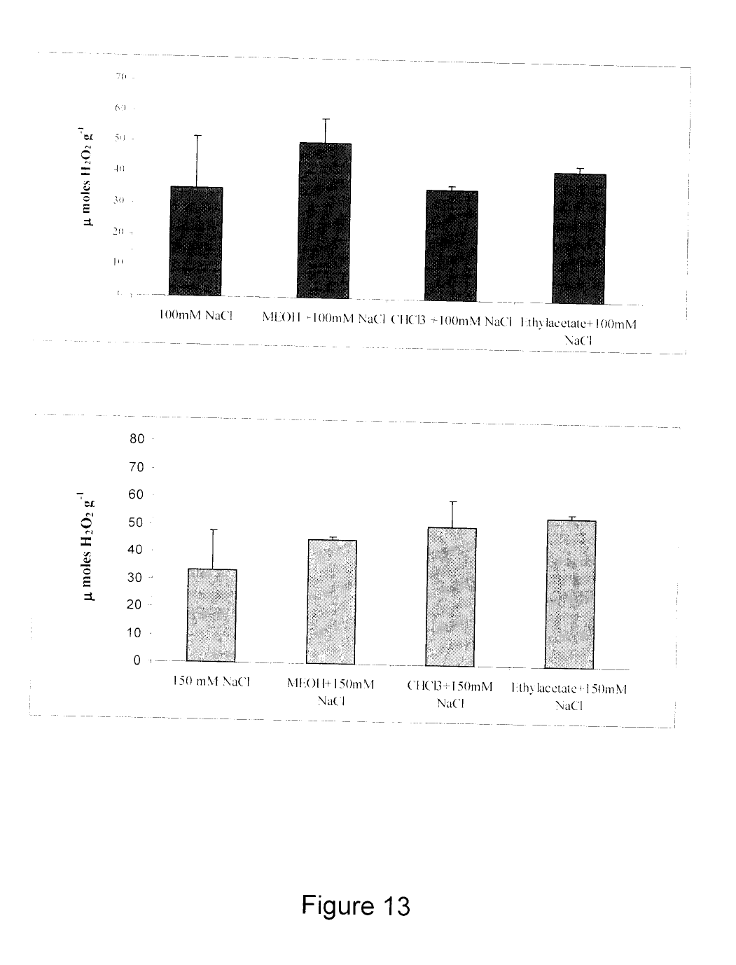

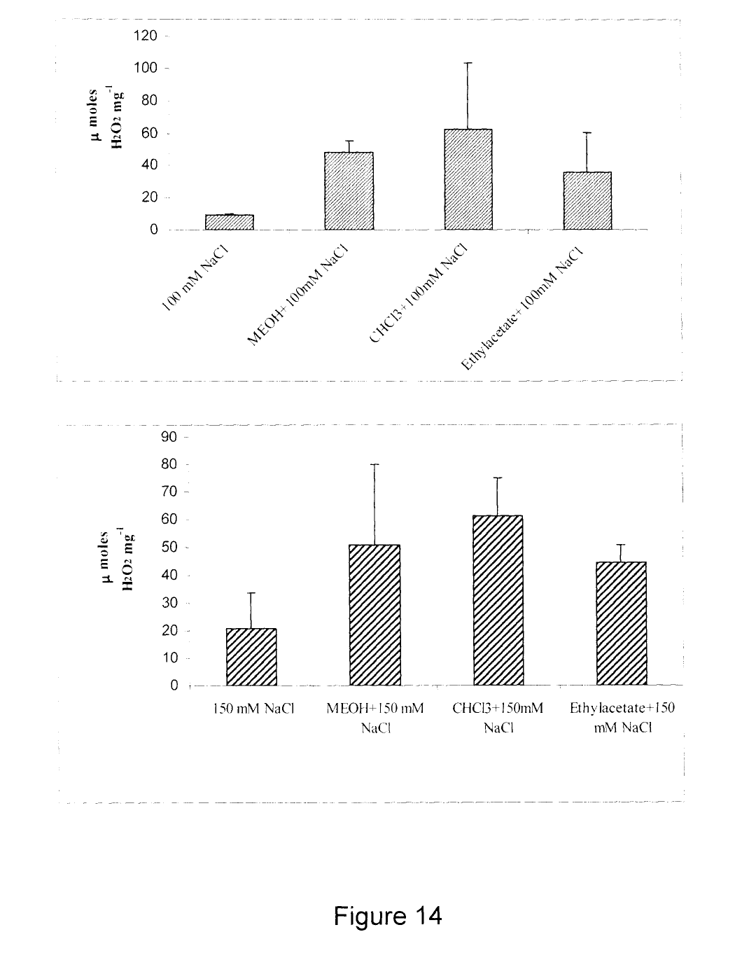

Catalase activity was assayed using the methodology described by Havir and McHale (1987). Ten to twenty discs were cut from the tip-half region of the fully expanded leaves with sharp punch. The leaves were washed in distilled water, randomized, and floated in groups of five (˜200 mg) in 30 ml control and treated solutions. The leaves were immediately placed in an ice-cold microfuge tube. To each microfuge tubes in the ice bath, 0.4 ml of freshly prepared ice cold buffer (potassium phosphate buffer, 50 mM at pH 7.4, containing 10 mm dithiothreitol) was added. Catalase enzyme was extracted by repeatedly inserting and rotating a tight-fitting plastic pestle into a microfuge tube for about 30 sec. The homogenate was centrifuged in a cold microcentrifuge at 12,000×g for 3 min. The tubes were stored in a ice bath. Catalase activity assay was carried out by adding 15 μl of the supernatant (crude enzyme extract) in 3.0 ml of assay medium (freshly prepared 12.5 mM hydrogen peroxide in 50 mM potassium phosphate, pH 7.0) in a 1 cm cuvette at 30° C. Catalase activity was measured by assaying the rate of decrease in absorbance at 240 nm to determine the initial rate (60 sec) of H₂O₂ breakdown. One unit is defined as the amount of enzyme catalyzing the decomposition of 1 μmol of hydrogen peroxide per minute under standard conditions at 30° C. Results are shown in FIGS. 13 and 14. As can be seen, extracts from A. nodosum enhance plant catalase activity in response to salt stress.

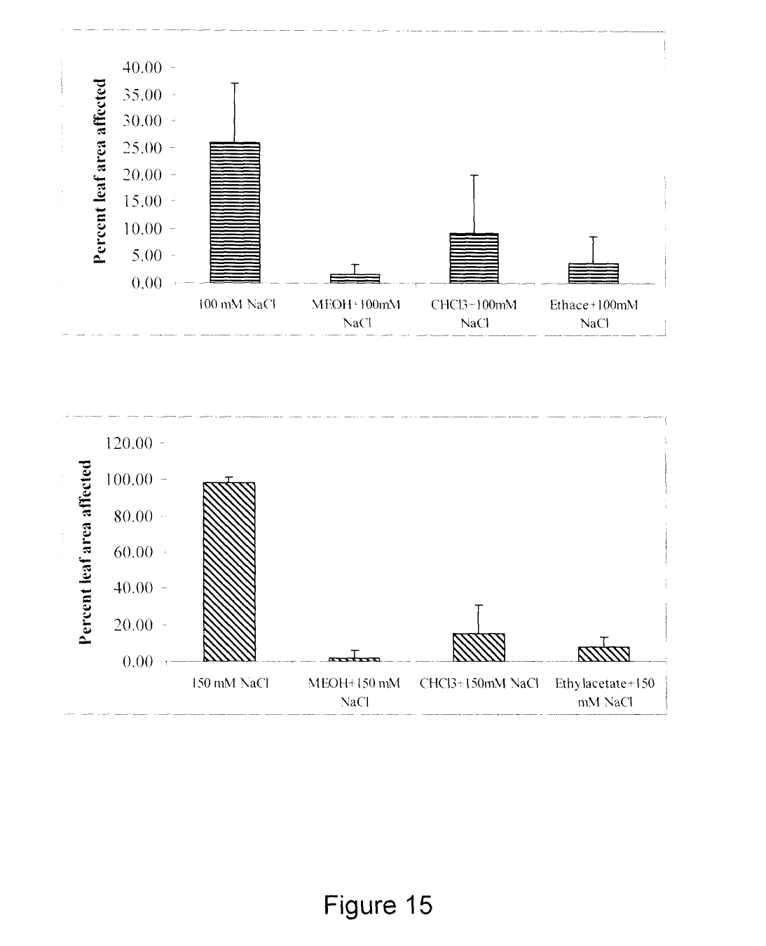

Percent leaf area: The percentage of leaf area affected by salt treatments in comparison with total leaf area was calculated using Sigma scan Pro® software package. Digital pictures of individual leaf bits in the treatments were calibrated in Sigma scan Pro® and leaf areas were measured. Results are shown in FIG. 15. As can be seen, extracts from A. nodosum reduce the percentage of leaf area affected by salt stress.

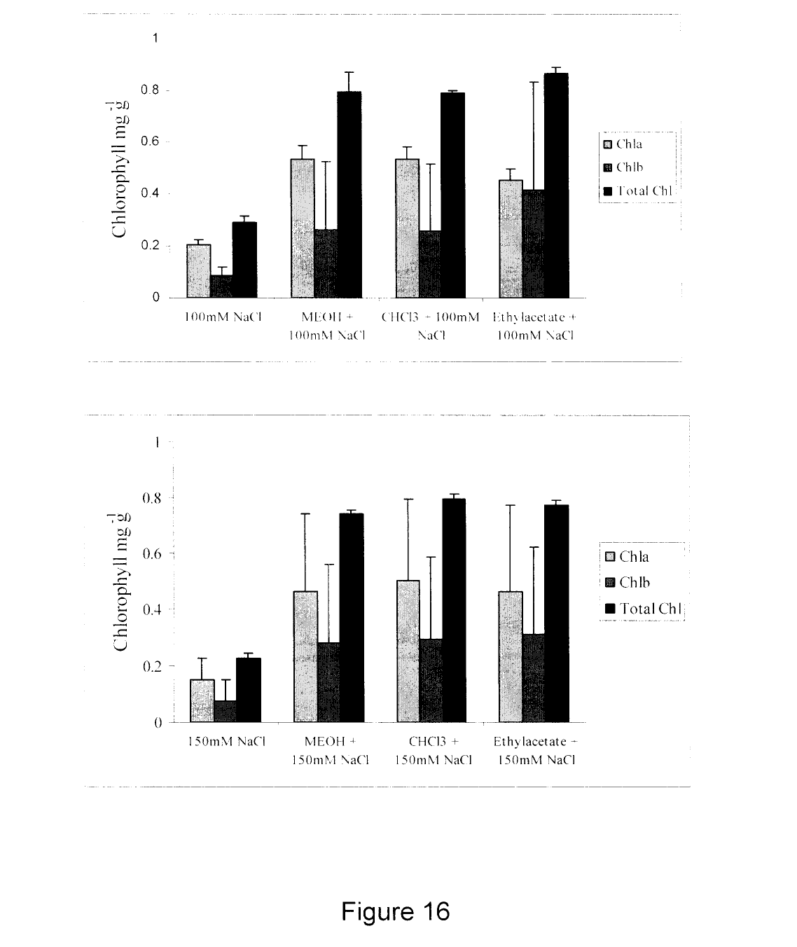

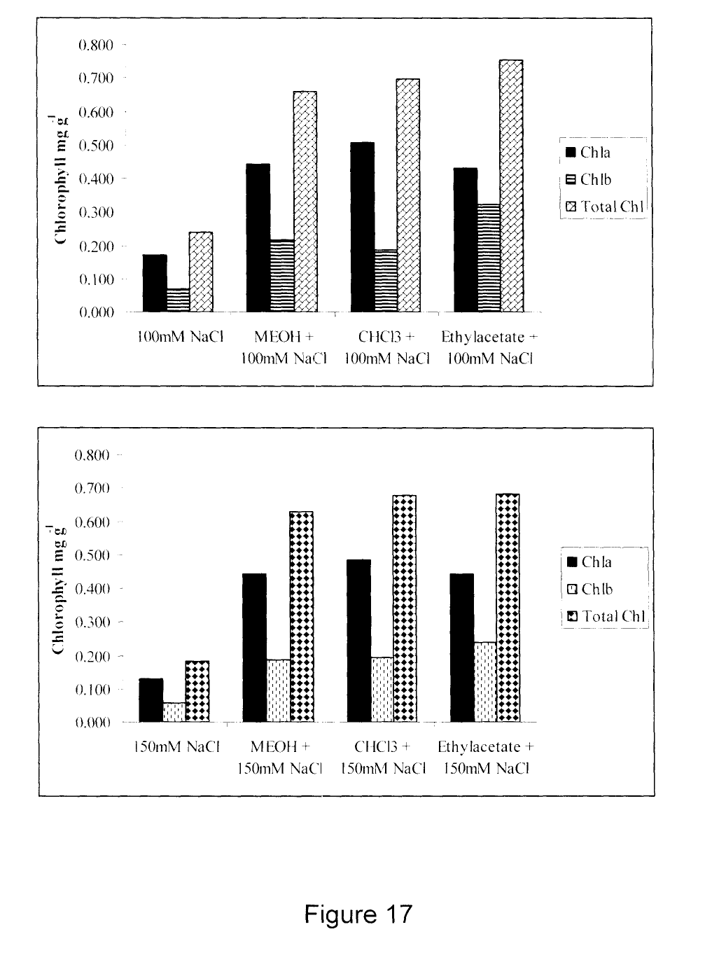

Estimation of chlorophyll content: Chlorophyll concentration in the leaf discs was measured using the protocol described by Arnon (1949). Approximately, 500 mg of the leaf discs per replication were taken and macerated with 80% acetone using a pestle and mortar. The macerated sample was then centrifuged at 3000 rpm for 10 min. The supernatant solution was decanted in a 25 ml volumetric flask and the volume was made up to 25 ml with 80% acetone. Using a spectrophotometer (Beckman Spectrophotometer Model; DU-65 S/n 20017), the optical density (OD) of the solution was recorded at 645 nm, 663 nm and 652 nm. The wavelength required for measurements was selected and the instrument was calibrated using a blank containing acetone at that respective wavelength before making measurements using the method described by Arnon, (1949). Results of measuring chlorophyll content in lettuce after 48 h under 100 mM NaCl and 150 mM NaCl conditions are shown in FIG. 16, and results of measuring chlorophyll content in sugarbeet after 48 h under 100 mM NaCl and 150 mM NaCl conditions are shown in FIG. 17. As can be seen, plants treated with extracts from A. nodosum retain chlorophyll levels even at 150 mM NaCl testing levels.

Effect of Fucosterol and Different Organic Extracts of A. nodosum on Root Length

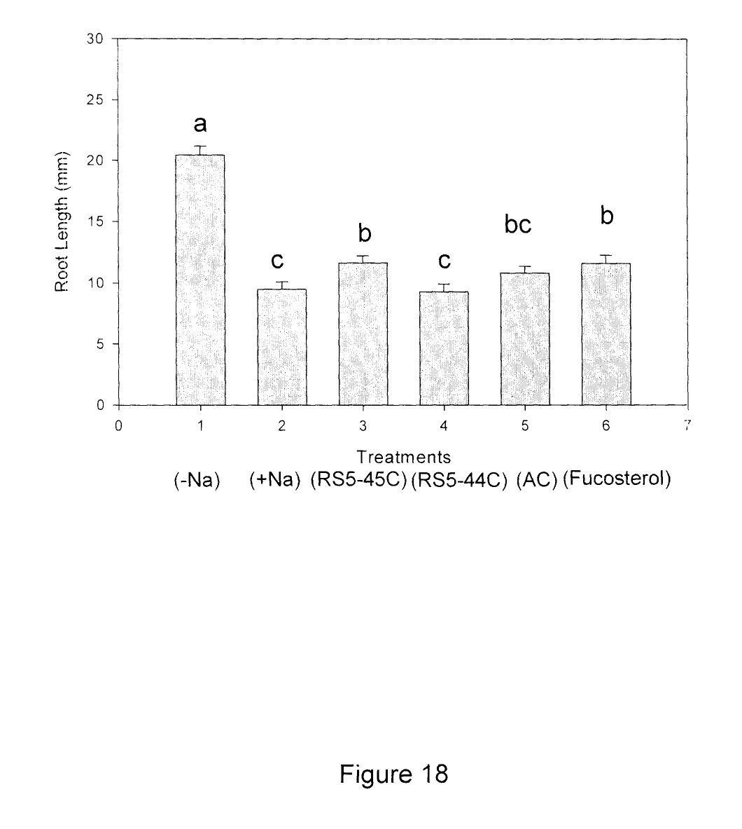

The code numbers in FIG. 18 refer to different organic extracts. As observed, plants treated with fucosterol and extract RS5-45C have a longer root length than untreated plants under salt stress (compared to Na⁺).

Ethyl Acetate Fraction Reduced Na⁺ Uptake by Roots

Ion Depletion Experiments: Net Na⁺ uptake and K⁺ loss from Arabidopsis roots was studied according to the method of Chen et al., 2007 (Chen C N, Chu C C, Zentella R, Pan S M, Ho T H (2002) AtHVA22 gene family in Arabidopsis: phylogenetic relationship, ABA and stress regulation, and tissue-specific expression. Plant Mol Biol 49: 633-644). Briefly, roots of 1 week-old Arabidopsis plants were immersed in glass vials containing 11 mL NaCl solution (80 mM NaCl, 0.5 mM KCl, and 0.1 mM CaCl₂) prepared using Millipore deionized water. Seedlings were kept at 25° C. in the dark for 24 h. At the end of incubation, roots were blotted dry, cut, and weighed. Na⁺ and K⁺ concentrations in the remaining solution were determined using Atomic absorption spectrophotometer, and net Na⁺ uptake and K⁺ loss were calculated on a fresh weight basis. This experiment was conducted with three replicates (5 plants per replicate).

NaCl treatments and Sodium and potassium estimation: Arabidopsis plants were grown as described above in a green house. For NaCl treatments, Arabidopsis plants grown in peat pellets were placed in individual plastic trays (5 cm diameter) at the rate of 15 plants. Each tray (constituting a replicate) was irrigated with 150 mM NaCl solution (prepared in distilled water) for 24 hours. For ethyl acetate fraction (EAA) treatments, 20 mL EAA was added subsequently. Additionally, the plants were irrigated on alternate days with distilled water to maintain uniform moisture for optimum growth of Arabidopsis plants. Control, NaCl treated plants received no EAA treatment, while, on the other hand, control plants received only water during the whole experiment. After the 5th day, leaf samples from two sets of replicates were harvested.

Arabidopsis leaf tissue (1 g) was flash frozen and ground in mortar and pestle. For Na⁺ and K⁺ ions measurement of ached leaf samples, method as described in AOAC 968.08 (Association of Offical Analytical Chemists, standard protocol) was performed using NaCl and KCl as standards. Briefly, 1 g of the ground leaf tissue was kept in a furnace, at 550° C. for 4 h. The samples were then cooled, 10 mL 3M HCl added and boiled gently for 10 mM. The solution was then filtered in a 100 mL volumetric flask, and diluted to final volume with deionized water. Subsequently, dilutions with 0.1-0.5M HCl was done to bring the samples in range with the NaCl and KCl standards.

Results: Differences in Na+ Uptake After Treatment with EAA Treatment

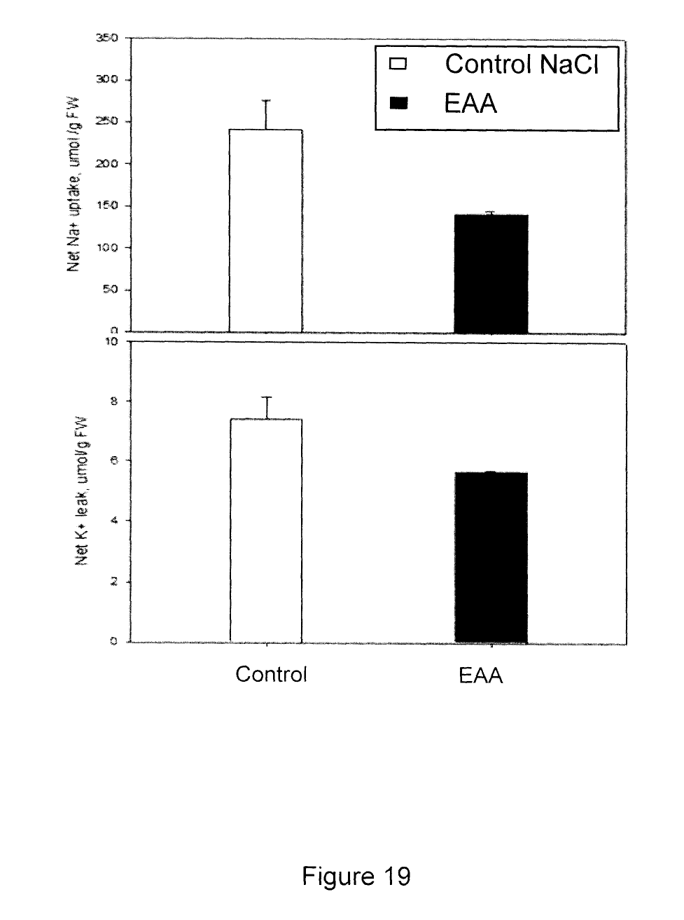

Plants exposed to high concentration of NaCl accumulate elevated concentration of Na⁺ in the tissue leading to disruption of ionic balance and ultimately cellular function. Arabidopsis plants were treated with EAA after 24 h exposure to 150 mM NaCl. As expected, sodium content in leaves of NaCl treated plants (150 mM) increased by 84% in comparison to control plants that were not irrigated with NaCl. Interestingly, plants treated with EAA caused a decreased accumulation of Na⁺ in the leaves; it reduced the concentration of Na⁺in the leaf tissue by 53%. Moreover, EAA treatments also decreased potassium content by 56%. On the other hand, nitrogen and phosphorous content differed only slightly between untreated and treated controls (see FIG. 9). Ion depletion experiments showed that Arabidopsis plants treated with EAA were not only able to reduce net root Na⁺ uptake by 41% compared with NaCl treated seedlings, but also loose 25% less K⁺ from the cytosol (FIG. 19).

It will be understood that numerous modifications to the above described invention will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims. All references cited herein are hereby incorporated by reference in their entirety.

1. A process for preparing an organic extract useful as a treatment for inducing salinity tolerance in plants, said process comprising the steps of:
suspending dried Ascophyllum nodosum or extracts of A. nodosum in methanol,
mixing the suspension, and
separating any remaining solid material from the resulting methanol extract,
wherein said methanol extract is useful as a treatment for inducing salinity tolerance in plants.

2. The process of claim 1, further comprising the steps of:
removing the solvent from said methanol extract to form a dried residual,
resuspending the dried residual from said methanol extract in water,
adding chloroform to the resuspended residual from the methanol extract,
mixing and allowing the phases to separate into water and chloroform extracts,
collecting the chloroform extract, and
removing the solvent from said chloroform extract to form a dried residual,
wherein the dried residual of said chloroform extract is useful as a treatment for inducing salinity tolerance in plants.

3. The process of claim 2, further comprising the steps:
resuspending the dried residual from said chloroform extract in water,
adding ethyl acetate to the resuspended residual from the chloroform extract,
mixing and allowing the phases to separate into water and ethyl acetate extracts,
collecting the ethyl acetate extract, and
removing the solvent from said ethyl acetate extract to form a dried residual,
wherein the dried residual of said ethyl acetate extract is useful as a treatment for inducing salinity tolerance in plants.

4. The process of claim 1, wherein the A. nodosum is suspended in the methanol in a ratio of A. nodosum to methanol from about 1:1 to about 1:50 volume/volume.

5. The process of claim 4, wherein the A. nodosum is suspended in the methanol in a ratio of A. nodosum to methanol of 1:3 volume/volume.

6. The process of claim 2, wherein the steps of adding chloroform to the resuspended residual from the methanol extract, mixing and allowing the phases to separate into water and chloroform extracts, and collecting the chloroform extract, are repeated up to 3 times.

7. The process of claim 3, wherein the steps of adding ethyl acetate to the resuspended residual from the chloroform extract, mixing and allowing the phases to separate into water and ethyl acetate extracts, and collecting the ethyl acetate extract, are repeated up to 3 times.

8. (canceled)

9. (canceled)

10. A composition for inducing salinity tolerance and/or for treating or ameliorating salinity stress in plants, comprising as active agent at least one phytosterol, fungal sterol, terpenoid or fatty acid, or combinations thereof, derived from Ascophyllum nodosum.

11. The composition according to claim 10, wherein said fungal sterol is derived from Mycosphaerella ascophylli.

12. The composition according to claim 10, wherein said phytosterol is fucosterol.

13. A method of inducing salinity tolerance and/or for treating or ameliorating salinity stress in plants, comprising administering a composition as defined in claim 10 to a plant in an amount effective to induce salinity tolerance and/or treat or ameliorate said salt salinity stress in said plant.

14. (canceled)

15. A method of inducing salinity tolerance and/or treating or ameliorating salinity stress in plants, comprising extracting at least one phytosterol, fungal sterol, terpenoid or fatty acid, or combinations thereof from Ascophyllum nodosum, and administering said organic extract to a plant in an amount effective to induce salinity tolerance and/or treat or ameliorate said salinity stress in said plant.

16. The method according to claim 15, wherein said fungal sterol is derived from Mycosphaerella ascophylli.

17. The method according to claim 15, wherein said phytosterol is fucosterol.

18. (canceled)

19. The composition according to claim 10, wherein said at least one phytosterol, fungal sterol, terpenoid, fatty acid, or combinations thereof elicit coordinated expression of multiple genes in said plant to induce salinity tolerance.

20. (canceled)

21. (canceled)

22. The composition according to claim 10, wherein said composition is obtained according to a process comprising the steps of:
suspending dried A. nodosum or extracts of A. nodosum in methanol,
mixing the suspension, and
separating any remaining solid material from the resulting methanol extract.

23. The composition of claim 22, wherein the methanol extract is further processed by the steps of:
removing the solvent from said methanol extract to form a dried residual,
resuspending the dried residual from said methanol extract in water,
adding chloroform to the resuspended residual from the methanol extract,
mixing and allowing the phases to separate into water and chloroform extracts,
collecting the chloroform extract, and
removing the solvent from said chloroform extract to form a dried residual.

24. The composition of claim 23, wherein the dried residual of said chloroform extract is further processed by the steps:
resuspending the dried residual from said chloroform extract in water,
adding ethyl acetate to the resuspended residual from the chloroform extract,
mixing and allowing the phases to separate into water and ethyl acetate extracts,
collecting the ethyl acetate extract, and
removing the solvent from said ethyl acetate extract to form a dried residual.

25. The composition of claim 22, wherein the A. nodosum is suspended in the methanol in a ratio of A. nodosum to methanol from about 1:1 to about 1:50 volume/volume.

26. The composition of claim 25, wherein the A. nodosum is suspended in the methanol in a ratio of A. nodosum to methanol of about 1:3 volume/volume.

27. The composition of claim 23, wherein the steps of adding chloroform to the resuspended residual from the methanol extract, mixing and allowing the phases to separate into water and chloroform extracts, and collecting the chloroform extract, are repeated up to 3 times.

28. The composition of claim 24, wherein the steps of adding ethyl acetate to the resuspended residual from the chloroform extract, mixing and allowing the phases to separate into water and ethyl acetate extracts, and collecting the ethyl acetate extract, are repeated up to 3 times.

29. The composition according to claim 10, formulated as a liquid for spray or root irrigation or as a solid for seed treatment.

30. A liquid formulation for spray or root irrigation useful for inducing salinity tolerance and/or treating or ameliorating salinity stress in plants, said formulation comprising a composition as defined in claim 10 and an acceptable carrier.

31. A solid formulation for seed treatment useful for inducing salinity tolerance and/or treating or ameliorating salinity stress in plants, said formulation comprising a composition as defined in claim 10 and an acceptable carrier.

32. The method of claim 15, wherein the organic extract is prepared according to the method of claim 1.
